(19) United States
(12) Patent Application Publication
(10) Pub. No.: US 2021/0338683 A1
(43) Pub. Date: Nov. 4, 2021

(54) TREATMENT OF VIRAL INFECTIONS WITH COMBINATION OF PIKFYVE KINASE INHIBITORS AND TMPRSS-2 INHIBITORS

(71) Applicant: AcuraStem, Inc., Monrovia, CA (US)

(72) Inventors: Justin K. Ichida, Monrovia, CA (US); Matthew Stremlau, Monrovia, CA (US); Hongyan Zhou, Monrovia, CA (US)

(21) Appl. No.: 17/302,438

(22) Filed: May 3, 2021

Related U.S. Application Data
(60) Provisional application No. 63/018,853, filed on May 1, 2020.

(51) Int. Cl. A61K 31/5377; A61K 31/155; A61K 31/245; A61P 31/14

(52) U.S. Cl. CPC A61K 31/5377; A61P 31/14; A61K 31/245; A61K 31/155

(57) ABSTRACT

The present invention relates to methods of treating viral infections including COVID-19 and compositions with a combination of (i) an inhibitor of phosphatidylinositol-3-phosphate 5-kinase (PIKfyve) and (ii) an inhibitor of transmembrane serine proteinase 2 (TMPRSS-2).

This application claims the benefit of U.S. Provisional Application No. 63/018,853, filed May 1, 2020, which is hereby incorporated by reference.

### FIELD OF THE INVENTION

The present invention relates to methods of treating viral infections including COVID-19 and compositions with a combination of (i) an inhibitor of phosphatidylinositol-3-phosphate 5-kinase (PIKfyve) and (ii) an inhibitor of transmembrane serine proteinase 2 (TMPRSS-2).

### BACKGROUND OF THE INVENTION

COVID-19 is caused by the coronavirus SARS-CoV-2.

Hoffmann et al., 2020, Cell 181, 271-280 report that SARS-CoV-2 infection depends on the host cell factors ACE2 and TMPRSS2.

A news release, https://www.eurekalert.org/pub_releases/2020-03/tiom-nie032420.php (accessed Apr. 28, 2020) states that nafamostat mesylate (brand name: Fusan), which is the drug used to treat acute pancreatitis, may effectively block the requisite viral entry process the new coronavirus (SARS-CoV-2) uses to spread and cause disease (COVID-19).

There remains a dire need for improved treatments for viral infections, including COVID-19.

### SUMMARY OF THE INVENTION

The present invention is directed to the treatment of viral infections with (i) an inhibitor of phosphatidylinositol-3-phosphate 5-kinase (PIKfyve) and (ii) an inhibitor of transmembrane serine proteinase 2 (TMPRSS-2).

One embodiment is a method for treating a subject having a viral infection comprising administering to the subject an effective amount of (i) a PIKfyve inhibitor and (ii) a TMPRSS-2 inhibitor. The subject can be a human subject. The viral infection can be caused by any type of virus such as RNA and DNA viruses. In one embodiment, the viral infection is caused by a coronavirus, such as SARS-CoV-2. The virus can also be African swine flu, pox virus (vaccinia virus or other pox virus), Ebola virus, middle east respiratory syndrome virus (MERS), JC polyomavirus (JC), BK polyomavirus (BK), Herpes Simplex Virus (HSV), Marburg virus (MarV), Venezuelan equine encephalitis virus (VEEV), or Lymphocytic choriomeningitis virus (LCMV).

In one embodiment the coronavirus is selected from human coronavirus 229E (alpha coronavirus), NL63 (alpha coronavirus), OC43 (beta coronavirus), and HKU1 (beta coronavirus).

In another embodiment, the coronavirus is MERS-CoV (the beta coronavirus that causes Middle East Respiratory Syndrome, or MERS).

In yet another embodiment, the coronavirus is SARS-CoV (the beta coronavirus that causes severe acute respiratory syndrome, or SARS).

In yet another embodiment, the coronavirus is SARS-CoV-2 (the novel coronavirus that causes coronavirus disease 2019, or COVID-19). In one embodiment, the human subject suffers from COVID-19. The SARS-CoV-2 can be selected from one of the following 10 Clades of SARS-CoV-2 (viruses with common ancestor): A1a, A2, A2a, A3, A6, A7, B, B1, B2, and B4. In one embodiment, the human subject suffers from COVID-19 and is experiencing mild symptoms, such as no or mild pneumonia. In another embodiment, the human subject suffers from severe COVID-19 (e.g., with dyspnea, hypoxia, or >50 percent lung involvement on imaging within 24 to 48 hours). In yet another embodiment, the human subject suffers from critical COVID-19 (e.g., with respiratory failure, shock, or multiorgan dysfunction).

In one preferred embodiment, the TMPRSS-2 inhibitor is nafamostat or a pharmaceutically acceptable salt thereof (e.g., nafamostat mesylate). The nafamostat or pharmaceutically acceptable salt thereof may be administered as a continuous intravenous infusion. In another embodiment, the TMPRSS-2 inhibitor is camostat or a pharmaceutically acceptable salt thereof. In one embodiment, about 2 to about 4 grams of camostat is administered per person per course.

In one embodiment, the PIKfyve inhibitor is selected from apilimod or a pharmaceutically acceptable salt thereof, APY0201, and YM-201636. In one preferred embodiment, the PIKfyve inhibitor is apilimod or a pharmaceutically acceptable salt thereof.

Another embodiment is a pharmaceutical composition comprising (i) a PIKfyve inhibitor and (ii) a TMPRSS-2 inhibitor. The pharmaceutical composition may further include ore or more pharmaceutically acceptable excipients.

In one embodiment, the method further comprises administering at least one additional active agent to the subject in a therapeutic regimen comprising a compound of the present invention and the at least one additional active agent. In one embodiment, the at least one additional active agent is selected from selected from the group consisting of apilimod, APY0201, YM-201636, remdesivir, favipiravir, and any combination of any of the foregoing. In one embodiment, the remdesivir is administered intravenously 200 mg on day 1, then 100 mg per day for another 5 to 10 days (for example, 7 days). The total dosing amount of remdesivir over the entire regimen can be about 1 gram per patient per course. In another embodiment, favipiravir is administered at about 3.2 to 4 grams per day (e.g., 3.6 grams per day).

In accordance with any of the methods described herein, a compound of the present invention may also be administered in combination with a non-therapeutic agent which mitigates one or more side effects associated with the compound or increases the bioavailability of the compound. In one embodiment, the non-therapeutic agent is selected from the group consisting of ondansetron, granisetron, dolasetron and palonosetron. In another aspect, the non-therapeutic agent is selected from the group consisting of pindolol and risperidone. In another aspect, the non-therapeutic agent is selected from a cytochrome P450 3A (CYP3A) inhibitor. In one embodiment, the CYP3A inhibitor is selected from ritonavir and cobicistat.

In the methods described here, the PIKfyve inhibitors and TMPRSS2 inhibitors can be administered by any suitable route, such as an oral, intravenous, or subcutaneous route.

### BRIEF DESCRIPTION OF THE FIGURES

FIG. 1 is a diagram showing two pathways by which SARS-CoV-2 enters cells.

FIG. 2 are graphs for percent relative inhibition and percent cytotoxicity for the ability of a Pikfyve inhibitor Compound A to block live SARS-CoV-2 viral entry in Vero E6 cells.

FIG. 3 is a plot showing the percent relative inhibition of viral infection with SARS-CoV-2 pseudovirus after treatment with one of ten Pikfyve antisense oligonucleotides.

FIG. 4 are graphs showing the percent relative inhibition of viral infection with SARS-CoV-2 pseudovirus after treatment with the Pikfyve inhibitor Compound A, the TMPRSS2 inhibitor nafamostat, or a combination of Compound A and nafamostat.

### DETAILED DESCRIPTION OF THE INVENTION

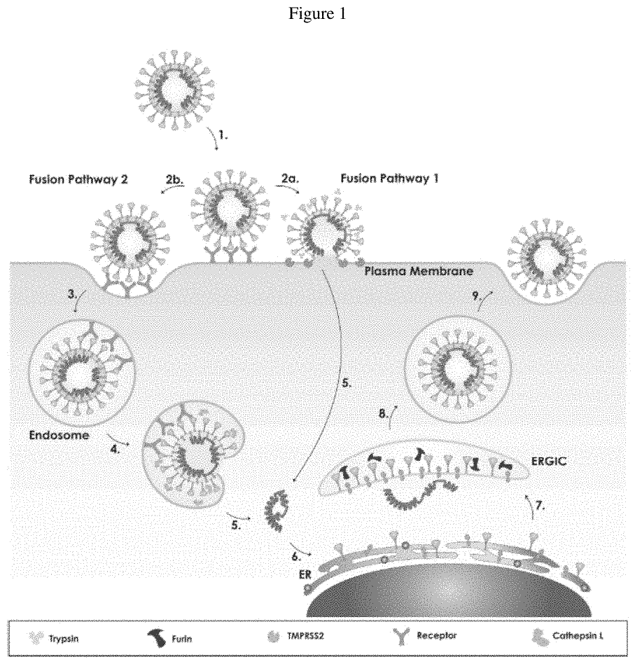

Without being bound by any particular theory, there are two routes by which SARS-CoV-2 enters cells: (1) the EARLY fusion pathway (Fusion Pathway #1) and (2) the LATE fusion pathyway (Fusion Pathway #2) as shown in FIG. 1. The EARLY fusion pathway (Fusion Pathway #1) happens at the cell membrane and the viral RNA/replication complex gets delivered directly into the cytoplasm. The LATE pathway is via endocytosis. The choice of pathway is cell type specific.

If there are certain proteases present at the cell membrane, then entry will occur via the EARLY pathway. The proteases are necessary to cleave the SARS Spike protein to initiate fusion of the viral particle and the host cell membrane. If the proteases are not present, then the virus enters via the LATE pathway which is mediated by endocytosis. In VERO and 293T cells, the proteases are not present at the cell membrane and entry occurs via the LATE pathway. In lung cells, entry occurs via the EARLY pathway hence, a different pathway than for VERO cells.

The inventors theorize that PIKfyve is involved in various steps of the EARLY fusion pathway, including steps 5, 7, 8, and 9 shown in FIG. 1. PIKfyve is also involved in the LATE fusion pathway, particularly endosomal maturation, as shown in step 4 of FIG. 1.

TMPRSS2 inhibitors block the EARLY pathway. Without being bound by any particular theory, the inventors theorize that PIKfyve inhibitors will act synergistically with TMPRSS2 inhibitors to prevent entry of SARS-CoV-2 into cells.

### Definitions

As used herein the following definitions shall apply unless otherwise indicated. Further, many of the groups defined herein can be optionally substituted. The listing of substituents in the definition is exemplary and is not to be construed to limit the substituents defined elsewhere in the specification.

The term “alkyl”, unless otherwise specified, refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl). The term “C₁₋₆ alkyl” refers to an alkyl group as defined above having up to 6 carbon atoms. The term “C₁₋₃ alkyl” refers to an alkyl group as defined above having up to 3 carbon atoms.

In appropriate circumstances, the term “alkyl” refers to a hydrocarbon chain radical as mentioned above which is bivalent.

The term “alkenyl”, unless otherwise specified, refers to an aliphatic hydrocarbon group containing one or more carbon-carbon double bonds and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, and 2-butenyl. The term “C₂₋₆ alkenyl” refers to an alkenyl group as defined above having up to 6 carbon atoms. In appropriate circumstances, the term “alkenyl” refers to a hydrocarbon group as mentioned above which is bivalent.

The term “alkynyl”, unless otherwise specified, refers to a straight or branched chain hydrocarbyl radical having at least one carbon-carbon triple bond, and having in the range of 2 to up to 12 carbon atoms (with radicals having in the range of 2 to up to 10 carbon atoms presently being preferred) e.g., ethynyl, propynyl, and butnyl. The term “C₂₋₆ alkynyl” refers to an alkynyl group as defined above having up to 6 carbon atoms. In appropriate circumstances, the term “alkynyl” refers to a hydrocarbyl radical as mentioned above which is bivalent.

The term “alkoxy” unless otherwise specified, denotes an alkyl, cycloalkyl, or cycloalkylalkyl group as defined above attached via an oxygen linkage to the rest of the molecule. The term “substituted alkoxy” refers to an alkoxy group where the alkyl constituent is substituted (i.e., —O-(substituted alkyl). For example “alkoxy” refers to the group —O-alkyl, including from 1 to 8 carbon atoms of a straight, branched, cyclic configuration and combinations thereof attached to the parent structure through an oxygen atom. Examples include methoxy, ethoxy, propoxy, isopropoxy, cyclopropyloxy, and cyclohexyloxy. In appropriate circumstances, the term “alkoxy” refers to a group as mentioned above which is bivalent.

The term “cycloalkyl”, unless otherwise specified, denotes a non-aromatic mono or multicyclic ring system of about 3 to 12 carbon atoms such as cyclopropyl, cyclobutyl, cyclopentyl, and cyclohexyl. Examples of multicyclic cycloalkyl groups include perhydronaphthyl, adamantyl and norbornyl groups, bridged cyclic groups, and sprirobicyclic groups, e.g., spiro[4.4]non-2-yl. The term “C₃₋₆ cycloalkyl” refers to a cycloalkyl group as defined above having up to 6 carbon atoms.

The term “cycloalkylalkyl”, unless otherwise specified, refers to a cyclic ring-containing radical containing in the range of about 3 up to 8 carbon atoms directly attached to an alkyl group which is then attached to the main structure at any carbon from the alkyl group, such as cyclopropylmethyl, cyclobutylethyl, and cyclopentylethyl.

The term “cycloalkenyl”, unless otherwise specified, refers to cyclic ring-containing radicals containing in the range of about 3 up to 8 carbon atoms with at least one carbon-carbon double bond such as cyclopropenyl, cyclobutenyl, and cyclopentenyl. The term “cycloalkenylalkyl” refers to a cycloalkenyl group directly attached to an alkyl group which is then attached to the main structure at any carbon from the alkyl group.

The term “aryl”, unless otherwise specified, refers to aromatic radicals having in the range of 6 up to 20 carbon atoms such as phenyl, naphthyl, tetrahydronaphthyl, indanyl, and biphenyl.

The term “arylalkyl”, unless otherwise specified, refers to an aryl group as defined above directly bonded to an alkyl group as defined above, e.g., —CH₂C₆H₅ and —C₂H₅C₆H₅.

The term “heterocyclic ring”, unless otherwise specified, refers to a non-aromatic 3 to 15 member ring radical which consists of carbon atoms and at least one heteroatom selected from nitrogen, phosphorus, oxygen and sulfur. For purposes of this invention, the heterocyclic ring radical may be a mono-, bi-, tri- or tetracyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized. The heterocyclic ring radical may be attached to the main structure at any heteroatom or carbon atom.

The term “heterocyclyl”, unless otherwise specified, refers to a heterocylic ring radical as defined above. The heterocylcyl ring radical may be attached to the main structure at any heteroatom or carbon ring atom. In appropriate circumstances, the term “heterocyclyl” refers to a hydrocarbon chain radical as mentioned above which is bivalent.

The term “heterocyclylalkyl”, unless otherwise specified, refers to a heterocylic ring radical as defined above directly bonded to an alkyl group. The heterocyclylalkyl radical may be attached to the main structure at any carbon atom in the alkyl group. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “heteroaryl”, unless otherwise specified, refers to an optionally substituted 5 to 14 member aromatic ring having one or more heteroatoms selected from N, O, and S as ring atoms. The heteroaryl may be a mono-, bi- or tricyclic ring system. Examples of such “heteroaryl” radicals include, but are not limited to, oxazolyl, thiazolyl, imidazolyl, pyrrolyl, furanyl, pyridinyl, pyrimidinyl, pyrazinyl, benzofuranyl, indolyl, benzothiazolyl, benzoxazolyl, carbazolyl, quinolyl, isoquinolyl, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, 4-piperidonyl, pyrrolidinyl, pyridazinyl, oxazolinyl, oxazolidinyl, triazolyl, indanyl, isoxazolyl, isoxazolidinyl, morpholinyl, thiazolinyl, thiazolidinyl, isothiazolyl, quinuclidinyl, isothiazolidinyl, isoindolyl, indolinyl, isoindolinyl, octahydroindolyl, octahydroisoindolyl, decahydroisoquinolyl, benzimidazolyl, thiadiazolyl, benzopyranyl, tetrahydrofuryl, tetrahydropyranyl, thienyl, benzothienyl, thiamo holinyl, thiamorpholinyl sulfoxide, thiamo holinyl sulfone, dioxaphospholanyl, oxadiazolyl, chromanyl, and isochromanyl.

The term “5 or 6-membered heteroaryl” refers to a heteroaryl having 5- or 6-ring atoms. The term “5-6 or 6-5 membered bicyclic heteroaryl” refers to a bicyclic heteroaryl with a five-membered ring fused to a six-membered ring, where the 5-membered ring is bound to the rest of the molecule (referred as a “5-6 membered bicyclic heteroaryl”) or the 6-membered ring is bound to the rest of the molecule (referred as a “6-5 membered bicyclic heteroaryl”). The term “6-6 membered bicyclic heteroaryl” refers to a bicyclic heteroaryl with a six-membered ring fused to a another six-membered ring, where one of the 6-membered rings is bound to the rest of the molecule.

The heteroaryl ring radical may be attached to the main structure at any heteroatom or carbon atom. The term “substituted heteroaryl” also includes ring systems substituted with one or more oxide (—O—) substituents, such as pyridinyl N-oxides.

The term “heteroarylalkyl”, unless otherwise specified, refers to a heteroaryl ring radical as defined above directly bonded to an alkyl group. The heteroarylalkyl radical may be attached to the main structure at any carbon atom from alkyl group.

The term “cyclic ring” refers to a cyclic ring containing 3 to 10 carbon atoms.

The term “substituted” unless otherwise specified, refers to substitution with any one or any combination of the following substituents which may be the same or different and are independently selected from hydrogen, hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted heteroaryl, substituted or unsubstituted heteroarylalkyl, substituted or unsubstituted heterocyclic ring, substituted heterocyclylalkyl ring, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, ═N—NR^{x}R^{y}, —NR^{x}C(O)OR^{y}, —NR^{x}R^{y}, —NR^{x}C(O)R^{y}, —NR^{x}C(S)R^{y}—NR^{x}C(S)NR^{y}R^{z}, —SONR^{x}R^{y}, —SO₂NR^{x}R^{y}, —OR^{x}, —OR^{x}C(O)NR^{y}R^{z}, —OR^{x}C(O)OR^{y}, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, and —ONO₂, wherein R^{x}, R^{y} and R^{z} in each of the above groups can be hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted amino, substituted or unsubstituted heteroaryl, substituted or unsubstituted heteroarylalkyl, substituted or unsubstituted heterocyclic ring, or substituted heterocyclylalkyl ring, or any two of R^{x}, R^{y} and R^{z} may be joined to form a substituted or unsubstituted saturated or unsaturated 3-10 membered ring, which may optionally include heteroatoms which may be the same or different and are selected from O, NR^{x} (e.g., R^{x} can be hydrogen or C₁₋₆ alkyl) or S. Substitution or the combinations of substituents envisioned by this invention are preferably those that result in the formation of a stable or chemically feasible compound. The term stable as used herein refers to the compounds or the structure that are not substantially altered when subjected to conditions to allow for their production, detection and preferably their recovery, purification and incorporation into a pharmaceutical composition. The substituents in the aforementioned “substituted” groups cannot be further substituted. For example, when the substituent on “substituted alkyl” is “substituted aryl”, the substituent on “substituted aryl” cannot be “substituted alkenyl”.

The term “halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

The term “protecting group” or “PG” refers to a substituent that is employed to block or protect a particular functionality. Other functional groups on the compound may remain reactive. For example, an “amino-protecting group” is a substituent attached to an amino group that blocks or protects the amino functionality in the compound. Suitable amino-protecting groups include, but are not limited to, acetyl, trifluoroacetyl, tert-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable hydroxy-protecting groups include, but are not limited to, acetyl and silyl. A “carboxy-protecting group” refers to a substituent of the carboxy group that blocks or protects the carboxy functionality. Suitable carboxy-protecting groups include, but are not limited to, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, and nitroethyl. For a general description of protecting groups and their use, see T. W. Greene, Protective Groups in Organic Synthesis, John Wiley & Sons, New York, 1991.

Certain of the compounds described herein contain one or more asymmetric centers and can thus give rise to enantiomers, diastereomers, and other stereoisomeric forms that can be defined, in terms of absolute stereochemistry, as (R)- or (S)—. The present chemical entities, pharmaceutical compositions and methods are meant to include all such possible isomers, including racemic mixtures, optically pure forms and intermediate mixtures. Non-limiting examples of intermediate mixtures include a mixture of isomers in a ratio of 10:90, 13:87, 17:83, 20:80, or 22:78. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

A “leaving group or atom” is any group or atom that will, under the reaction conditions, cleave from the starting material, thus promoting reaction at a specified site. Suitable examples of such groups unless otherwise specified are halogen atoms and mesyloxy, p-nitrobenzensulphonyloxy and tosyloxy groups.

Additionally, the instant invention also includes the compounds which differ only in the presence of one or more isotopically enriched atoms for example replacement of hydrogen with deuterium or tritium, the replacement of a carbon by ¹³C- or ¹⁴C-enriched carbon, or the replacement of a nitrogen by ¹⁵N.

The compounds of the present invention may also contain unnatural proportions of atomic isotopes at one or more of atoms that constitute such compounds. For example, the compounds may be radiolabeled with radioactive isotopes, such as for example tritium, iodine-125 (¹²⁵J) or carbon-14 (¹⁴C). All isotopic variations of the compounds of the present invention, whether radioactive or not, are encompassed within the scope of the present invention.

Pharmaceutically acceptable salts forming part of this invention include salts derived from inorganic bases such as Li, Na, K, Ca, Mg, Fe, Cu, Zn, and Mn; salts of organic bases such as N,N′-diacetylethylenediamine, glucamine, triethylamine, choline, hydroxide, dicyclohexylamine, metformin, benzylamine, trialkylamine, and thiamine; chiral bases such as alkylphenylamine, glycinol, and phenyl glycinol; salts of natural amino acids such as glycine, alanine, valine, leucine, isoleucine, norleucine, tyrosine, cystine, cysteine, methionine, proline, hydroxy proline, histidine, ornithine, lysine, arginine, and serine; quaternary ammonium salts of the compounds of invention with alkyl halides, and alkyl sulphates. Salts may include acid addition salts where appropriate which are sulphates, nitrates, phosphates, perchlorates, borates, hydrohalides (e.g., hydrochlorides), acetates, tartrates, maleates, citrates, fumarates, succinates, palmoates, methanesulphonates, benzoates, salicylates, benzenesulfonates, ascorbates, glycerophosphates, and ketoglutarates. Salts can be formed by methods known in the art.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried. In one embodiment, the amount of PIKfyve inhibitor administered ranges from about 0.1 mg to 5 g, from about 1 mg to 2.0 g, from about 100 mg to 1.5 g, from about 200 mg to 1.5 g, from about 400 mg to 1.5 g, and from about 400 mg to 1.0 g. In another embodiment, the dose of PIKfyve inhibitor ranges from about 0.1 μg to 1 g/kg body weight per day. In one embodiment, the dose of TMPRSS-2 inhibitor ranges from about 0.01 μg to 1 g/kg body weight per day, such as from about 0.1 μg to 1 g/kg body weight per day.

As used herein, the term “treating” refers to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable excipient” includes, but is not limited to, any and all solvents, dispersion media, coatings, antibacterial and antifungal agents, isotonic and absorption delaying agents, one or more suitable diluents, fillers, salts, disintegrants, binders, lubricants, glidants, wetting agents, controlled release matrices, colorants/flavoring, carriers, buffers, stabilizers, solubilizers, and combinations thereof. Except insofar as any conventional media or agent is incompatible with the active ingredient, its use in the therapeutic compositions of the invention is contemplated. Supplementary active ingredients can also be incorporated into the compositions.

A “subject” includes a mammal. The mammal can be e.g., any mammal, e.g., a human, primate, vertebrate, bird, mouse, rat, fowl, dog, cat, cow, horse, goat, camel, sheep or a pig. Preferably, the mammal is a human. The term “patient” refers to a human subject.

In accordance with the methods described herein, a “subject in need of” is a subject having a disease, disorder or condition, or a subject having an increased risk of developing a disease, disorder or condition relative to the population at large. The subject in need thereof can be one that is “non-responsive” or “refractory” to a currently available therapy for the disease or disorder, for example cancer. In this context, the terms “non-responsive” and “refractory” refer to the subject's response to therapy as not clinically adequate to relieve one or more symptoms associated with the disease or disorder. In one aspect of the methods described here, the subject in need thereof is a subject having cancer whose cancer is refractory to standard therapy or whose cancer has recurred following standard treatment.

### TMPRSS-2 Inhibitors

Suitable TMPRSS-2 inhibitors include, but are not limited to, nafamostat, camostat, and pharmaceutically acceptable salts thereof (e.g., camostat mesylate) as well as those described in Meyer et al., Biochem J. (2013) 452, 331-343, Rensi et al., ChemRxic Mar. 20, 2020 (doi.org/10.26434/chemrxiv.12009582.v1), U.S. Patent Publication No. 2013/0273070, and International Publication Nos. WO 2013/014074 and WO 2010/149459, all of which are hereby incorporated by reference in their entireties. Other TMPRSS-2 inhibitors include, but are not limited to, argatroban, otamixaban, letaxaban, darexaban, edoxaban, and betrixaban. The inhibitor can be an anti-TMPRSS-2 antibody or antigen-binding fragment thereof.

### PIKfyve Inhibitors

Suitable PIKfyve inhibitors include, but are not limited to, apilimod as well as those described in International Publication No. WO 2019/046316, U.S. Provisional Patent Application No. 62/975,092, filed Feb. 11, 2020, and International Patent Application No. PCT/US21/70144, filed Feb. 11, 2021, all of which are hereby incorporated by reference in their entireties.

In one embodiment, the PIKfyve inhibitor is a compound of the formula (I):

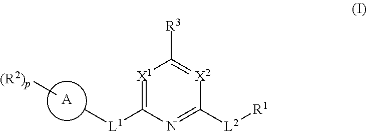

or a pharmaceutically acceptable salt thereof, wherein

R¹ is hydroxy, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl;

each occurrence of R² is independently substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, or substituted or unsubstituted heterocyclyl;

R³ is a nitrogen- or oxygen-containing moiety;

Ring A is (i) a 5 or 6-membered heteroaryl, a 5-6, 6-5 or 6-6 membered bicyclic heteroaryl, or a heterocyclyl, each having at least one nitrogen or oxygen ring atom, or (ii) phenyl;

L¹ is absent, C₁-C₂ alkylene, —NR^{c}—, —O—, —S—, —C(O)—, —NHC(O)—, —C(O)NH—, —NR^{c}C(O)—, or —NR^{c}C(O)(CR^{a}R^{b})ₘ—;

L² is —O—, —O—(CR^{a}R^{b})ₘ—, —(CR^{a}R^{b})ₘ—, —NR^{c}—(CR^{a}R^{b})ₘ—, or —S—(CR^{a}R^{b})ₘ—;

(i) X¹ is CH or CR^{c} and X² is N, or (ii) X¹ is N and X² is N, CH, or CR^{c};

each occurrence of R^{a} and R^{b} are independently hydrogen, hydroxy, hydroxy(C₁₋₄)alkyl, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl, halogen, nitro, —OR^{d}, —SR^{d}, —NR^{d}R^{e}, —C(O)R^{d}, —C(S)R^{d}, —OC(O)R^{d}, —SC(O)R^{d}, OC(S)R^{d}, SC(S)R^{d}, —NR^{c}C(O)R^{d}, —NR^{c}C(S)R^{d}, —SO₂R^{c}, —S(O)R^{c}, —NR^{c}SO₂R^{d}, —OS(O)₂R^{d}, —OP(O)R^{d}R^{e}, or —P(O)R^{d}R^{e};

each occurrence of R^{c} is independently a hydrogen or C₁₋₆ alkyl (e.g., C₁-C₄ alkyl);

each occurrence of R^{d} and R^{e} are independently hydrogen, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl;

each occurrence of m is independently 1-4; and

p is 1 or 2,

with the proviso that the compound of formula (I) possesses at least one of:

(i) Ring A is a 5-6, 6-5 or 6-6 membered bicyclic heteroaryl, or a heterocyclyl (such as a lactone or lactam),

(ii) L¹ is —NR^{c}C(O)— or —NR^{c}C(O)(CR^{a}R^{b})ₘ—,

(iii) L² is —O—, or

(iv) X² is CH or CR^{c}.

In one embodiment, R¹ is heterocyclyl or heteroaryl. For example, R¹ may be selected from (the squiggly lines indicate the point of attached to the rest of the molecule)

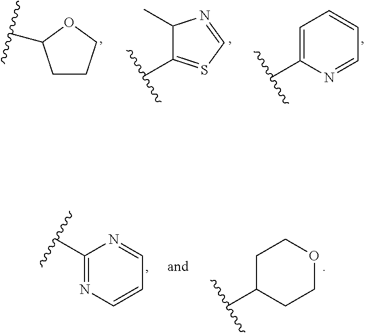

In another embodiment, R¹ is hydroxy.

In one embodiment, each occurrence of R² is independently substituted or unsubstituted aryl, such as a substituted or unsubstituted phenyl. For instance, R² may be phenyl, a halogen-substituted phenyl, an alkyl-substituted phenyl (e.g., a C₁₋₄ alkyl-substituted phenyl), a halogenated alkyl-substituted phenyl, or an alkoxy-substituted phenyl. In one embodiment, R² is selected from phenyl, 3-methoxyphenyl, 3-methylphenyl, 3-trifluoromethylphenyl, and 3-chlorophenyl. In a preferred embodiment, R² is selected from phenyl, 3-methoxyphenyl, and 3-methylphenyl.

In another embodiment, each occurrence of R² is independently substituted or unsubstituted alkyl (such as a C₁₋₄ alkyl). For instance, R² can be unsubstituted isopropyl.

In one embodiment, R³ is a substituted or unsubstituted, saturated or unsaturated nitrogen- or oxygen-containing heterocyclyl. For instance, R³ can be a substituted or unsubstituted, saturated or unsaturated 5-10 membered (such as a 5-8 membered) mono- or bi-cyclic heterocyclyl having at least one nitrogen or oxygen ring atom. In one embodiment, R³ is a substituted or unsubstituted 5-10 membered (such as a 5-8 membered) mono- or bi-cyclic heterocyclyl having at least one nitrogen atom and optionally an oxygen ring atom, where the nitrogen ring atom is directly attached to the rest of the molecule. In one preferred embodiment, R³ is a substituted or unsubstituted (unsaturated) 5-membered monocyclic heterocyclyl having an oxygen ring atom or a nitrogen ring atom.

In another embodiment, R³ is a substituted or unsubstituted, saturated or unsaturated 6-membered monocyclic heterocyclyl having an oxygen ring atom and optionally a nitrogen ring atom. In yet another embodiment, R³ is a saturated 8-membered bicyclic heterocyclyl having a nitrogen ring atom and an oxygen ring atom. In one embodiment, R³ is selected from

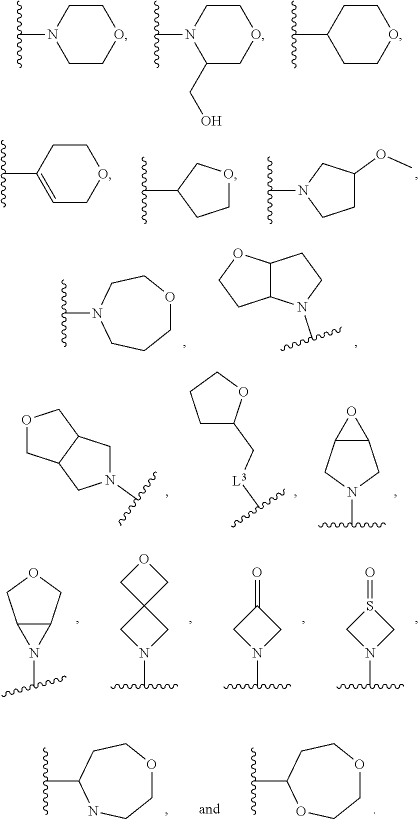

In one preferred embodiment, R³ is selected from

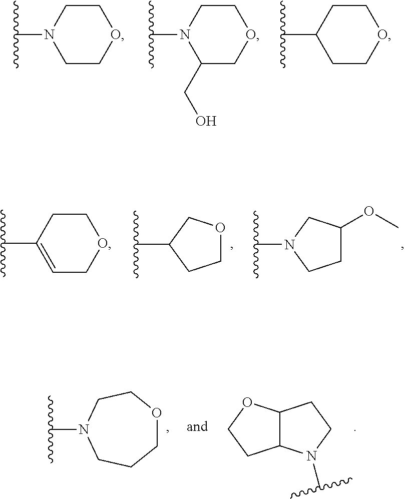

In another preferred embodiment, R³ is

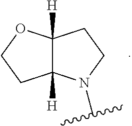 [SMILES: *N1CC[C@H]2OCC[C@H]21]

In yet another embodiment, R³ is a sulfonyl group of the formula —S(O)(CH₂)_{q}OR⁴, where R⁴ is hydrogen or C₁-C₄ alkyl and q is 1-4.

In one embodiment, ring A is a 5-membered heteroaryl having at least one nitrogen ring atom. In one preferred embodiment, ring A includes two heteroatoms as ring atoms (such as two nitrogen ring atoms, or one nitrogen ring atom with one sulfur ring atom). In another preferred embodiment, ring A is selected from

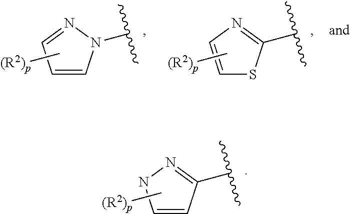

For instance, ring A can be selected from

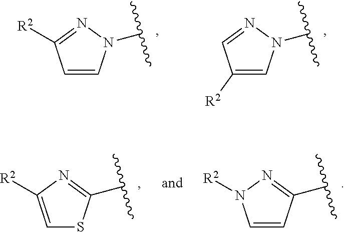

In one preferred embodiment, the R² group in ring A above is selected from substituted or unsubstituted aryl, such as a substituted or unsubstituted phenyl. For instance, R² may be phenyl, an alkyl-substituted, or an alkoxy-substituted phenyl. In a preferred embodiment, R² is selected from phenyl, 3-methoxyphenyl, and 3-methylphenyl.

In one embodiment, ring A is a heterocyclyl having at least one oxygen ring atom. In one preferred embodiment, the heterocyclyl is a lactone.

In one embodiment, ring A is a heterocyclyl having at least one nitrogen ring atom. In one preferred embodiment, the heterocyclyl is a lactam. In preferred embodiments, the lactam is a 5-membered lactam. In preferred embodiments, the 5-membered lactam is selected from

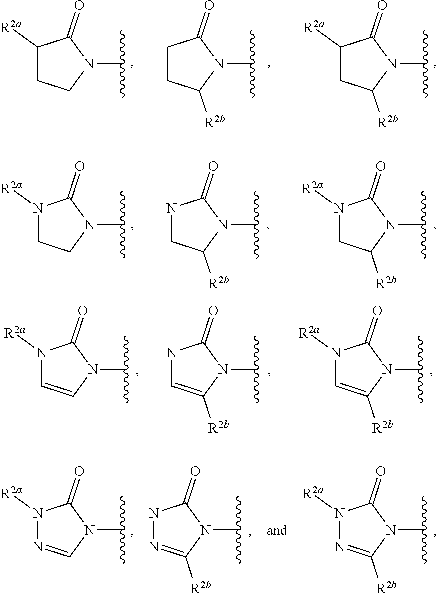

wherein each of R^{2a} and R^{2b} are independently selected from the R² groups listed above. In some instances, each of R^{2a} and R^{2b} are the same functional group. In some instances, R^{2a} and R^{2b} are different functional groups.

In one embodiment, L¹ is absent.

In another embodiment, L¹ is —NH—, —N(CH₃)—, —O—, or —CH₂—. In one embodiment, L¹ is —NH—. In another embodiment, L¹ is —C(O)NH— (where the carbonyl is attached to the rest of the molecule and the nitrogen is attached to ring A). In yet another embodiment, L¹ is —NHC(O)— (where the nitrogen atom is attached to the rest of the molecule and the carbonyl is attached to ring A).

In one embodiment, L² is —O—(CR^{a}R^{b})ₘ—. In one preferred embodiment, L² is —OCH₂CH₂- or —OCH₂—. In another embodiment, L² is —OCH₂CH₂CH(OH)CH₂—.

In another embodiment, L² is —(CR^{a}R^{b})ₘ—. In one preferred embodiment, L² is —CH₂CH₂—.

In yet another embodiment, L² is —NR^{c}—(CR^{a}R^{b})ₘ—, such as —NH—(CR^{a}R^{b})ₘ— (e.g., —NH—, —NHCH₂—, and —NHCH₂CH₂—).

In one embodiment, -L²-R¹ is —OCH₂CH₂CH(OH)CH₂OH.

In one preferred embodiment, X¹ is CH. In another embodiment, X¹ is N.

In one embodiment, each occurrence of R^{a} and R^{b} are independently hydrogen, hydroxy, or hydroxy(C₁₋₄)alkyl. In another embodiment, each occurrence of R^{a} and R^{b} are independently hydrogen or hydroxy.

In one embodiment, m is 1. In another embodiment, m is 2. In a preferred embodiment, m is 1 or 2 when R¹ is cyclic. In another preferred embodiment, m is 3 or 4 when R¹ is acyclic.

In a preferred embodiment, p is 1.

In another embodiment, p is 2.

In one preferred embodiment, the moiety

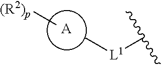

is selected from

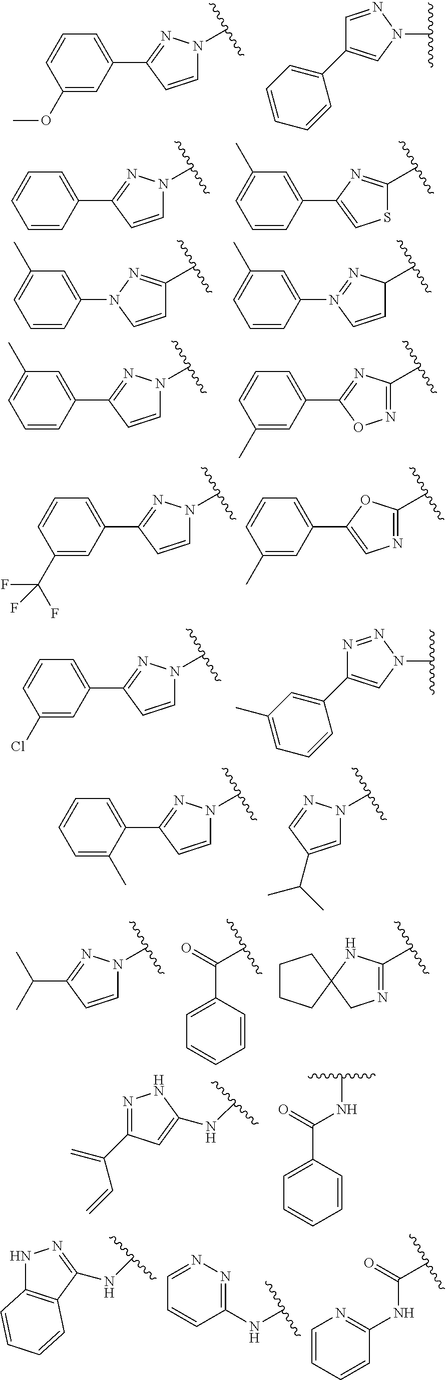

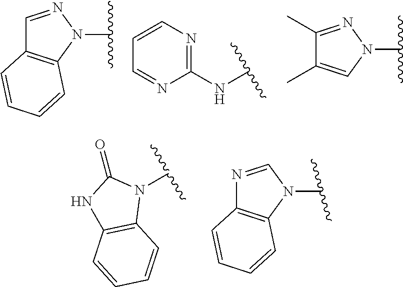

Another embodiment is a compound of the formula II

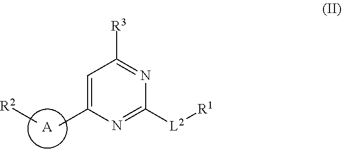

or a pharmaceutically acceptable salt thereof, wherein

R¹ is hydrogen, hydroxy, substituted or unsubstituted heteroaryl, or substituted or unsubstituted heterocyclyl;

R² is substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, or substituted or unsubstituted heterocyclyl;

R³ is a substituted or unsubstituted oxygen-containing heterocyclyl;

Ring A is a 5-membered heteroaryl having at least one nitrogen ring atom, or a 5-membered heterocyclyl having at least one nitrogen ring atom or one oxygen ring atom (e.g., a lactam or lactone);

L² is absent, —O—(CR^{a}R^{b})ₘ—, —O—, —CH₂—, —CHR^{a}—, —NH—, —NR^{a}—, —C(O)—, —NHC(O)—, —C(O)NH—, or a 5-membered heterocyclyl having at least one nitrogen ring atom or one oxygen ring atom (e.g., a lactam or lactone);

each occurrence of R^{a} and R^{b} are independently hydrogen, hydroxy, or hydroxy(C₁₋₄)alkyl; and

m is 1-4,

with the proviso that the compound of the formula (II) possesses at least one of:

(i) Ring A is a 5-membered heterocyclyl having at least one nitrogen ring atom or one oxygen ring atom; and

(ii) L² is —CH₂—, —CHR^{a}—, —NH—, —NR^{a}—, —C(O)—, —NHC(O)—, C(O)NH—, or a 5-membered heterocyclyl having at least one nitrogen ring atom or one oxygen ring atom.

In one embodiment of the compound of formula (II), R¹ is heterocylyl or heteroaryl. For example, R¹ may be selected from

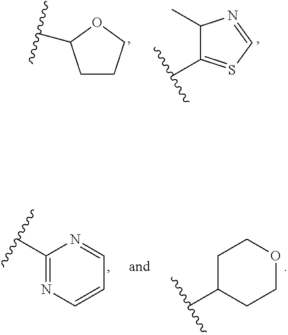

In one embodiment of the compound of formula (II), R² is substituted phenyl, such as an alkoxy-substituted phenyl, halogen-substituted phenyl, or alkyl-substituted phenyl. For example, R² can be methoxyphenyl (e.g., 3-methoxyphenyl) or methylphenyl (e.g., 3-methylphenyl).

In another embodiment of the compound of formula (II), R² is hydroxy.

In one preferred embodiment of the compound of formula (II), R³ is selected from

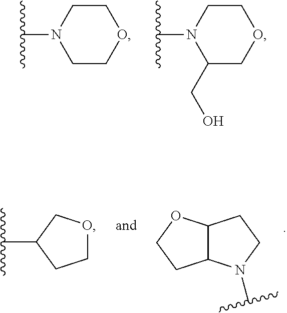

In another preferred embodiment, R³ is

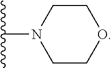 [SMILES: *N1CCOCC1]

In another preferred embodiment, R³ is

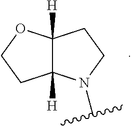 [SMILES: *N1CC[C@H]2OCC[C@H]21]

In one embodiment of the compound of formula (II), ring A is a 5-membered heteroaryl having (i) two nitrogen ring atoms or (ii) one nitrogen ring atom and one sulfur ring atom. In another embodiment, ring A is selected from

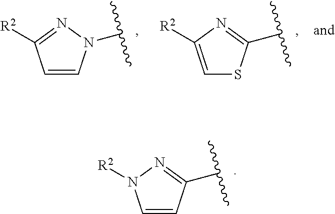

In another embodiment of the compound of formula (II), ring A is a 5-membered lactone.

In another embodiment of the compound of formula (II), ring A is a 5-membered lactam.

In preferred embodiments, the 5-membered lactam is selected from

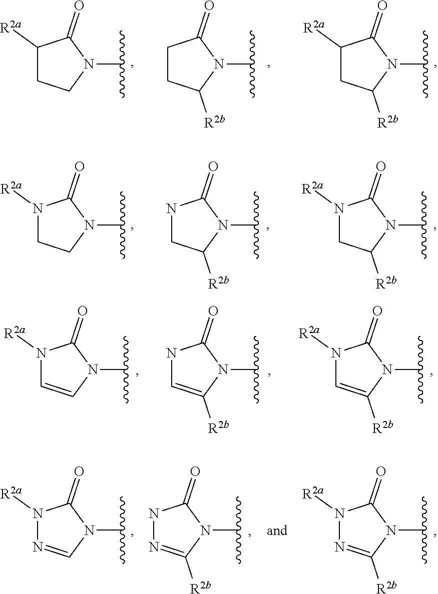

wherein each of R^{2a} and R^{2b} are independently selected from the R² groups listed above. In some instances, each of R^{2a} and R^{2b} are the same functional group. In some instances, R^{2a} and R^{2b} are different functional groups.

In one embodiment of the compound of formula (II), L² is —OCH₂—, —OCH₂CH₂—, —OCH₂CH₂CH(OH)CH₂—, or —CH₂CH₂—. In one preferred embodiment, L² is —OCH₂—, —OCH₂CH₂— or —OCH₂CH₂CH(OH)CH₂—.

In another embodiment of the compound of formula (II), L² is a 5-membered lactone. In another embodiment of the compound of formula (II), L² is a 5-membered lactam.

In yet another embodiment of the compound of formula (II), L² is —CH₂—, —CHR^{a}—, —NH—, —NR^{a}—, —C(O)—, —NHC(O)—, —C(O)NH—, or a 5-membered heterocyclyl having at least one nitrogen ring atom or one oxygen ring atom (e.g., a lactam or lactone).

Yet another embodiment is a compound of the formula (III):

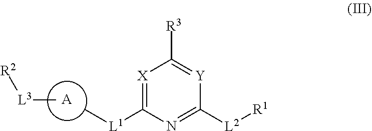

or a pharmaceutically acceptable salt thereof, wherein

R¹ is hydroxy, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl;

R² is substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, or substituted or unsubstituted heterocyclyl;

R³ is a nitrogen- or oxygen-containing moiety;

L¹ is —CH(CH₃)—, —CH₂CH(CH₃)—, —CH(CH₃)CH₂—, —N(CH₃)—, —N(CH₃)C(O)—, C(O)N(CH₃)—, or —N(CH₃)C(O)(CR^{a}R^{b})ₘ;

L² is —O—(CR^{a}R^{b})ₘ—, —(CR^{a}R^{b})ₘ—, —NR^{c}—(CR^{a}R^{b})ₘ—, or —S—(CR^{a}R^{b})ₘ—, —CH₂—, —CHR^{a}—, —NH—, —NR^{a}—, —C(O)—, —NHC(O)—, C(O)NH—, or a 5-membered heterocyclyl having at least one nitrogen ring atom or one oxygen ring atom;

L³ is —O—(CR^{a}R^{b})ₘ—, —(CR^{a}R^{b})ₘ—, —NR^{c}—(CR^{a}R^{b})ₘ—, or —S—(CR^{a}R^{b})ₘ—, —CH₂—, —CHR^{a}—, —NH—, —NR^{a}—, —C(O)—, —NHC(O)—, C(O)NH—, or a 5-membered heterocyclyl having at least one nitrogen ring atom or one oxygen ring atom;

(i) X is CH or CR^{c} and Y is N, or (ii) X is N and Y is N, CH, or CR^{c};

Ring A is (i) a 5 or 6-membered heteroaryl, a 5-6, 6-5 or 6-6 membered bicyclic heteroaryl, or a heterocyclyl, each having at least one nitrogen or oxygen ring atom, or (ii) phenyl;

each occurrence of R^{a} and R^{b} are independently hydrogen, hydroxy, hydroxy(C₁₋₄)alkyl, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl, halogen, nitro, —OR^{d}, —SR^{d}, —NR^{d}R^{e}, —C(O)R^{d}, —C(S)R^{d}, —OC(O)R^{d}, —SC(O)R^{d}, OC(S)R^{d}, SC(S)R^{d}, —NR^{c}C(O)R^{d}, —NR^{c}C(S)R^{d}, —SO₂R^{c}, —S(O)R^{c}, —NR^{c}SO₂R^{d}, —OS(O)₂R^{d}, —OP(O)R^{d}R^{e}, or —P(O)R^{d}R^{e};

R^{c} is a hydrogen or C₁₋₆ alkyl (e.g., C₁-C₄ alkyl);

each occurrence of R^{d} and R^{e} are independently hydrogen, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl; and

each occurrence of m is independently 1-4.

In one preferred embodiment of the compound of formula (III), R³ is selected from

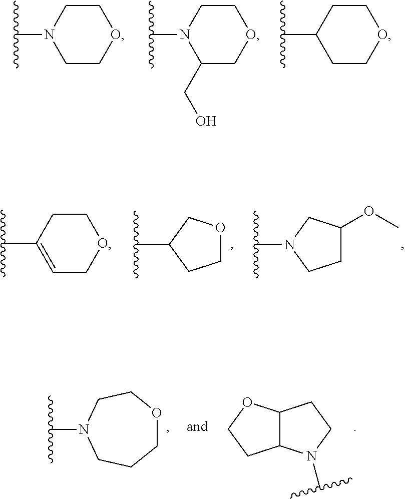

In another preferred embodiment, R³ is

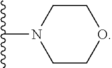 [SMILES: *N1CCOCC1]

In another preferred embodiment, R³ is

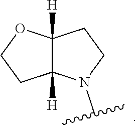 [SMILES: *N1CC[C@H]2OCC[C@H]21]

In yet another embodiment, R³ is a sulfonyl group of the formula —S(O)(CH₂)_{q}OR⁴, where R⁴ is hydrogen or C₁-C₄ alkyl and q is 1-4.

In another embodiment of the compound of formula (III), L² is a 5-membered lactone.

In another embodiment of the compound of formula (III), L² is a 5-membered lactam.

In another embodiment of the compound of formula (III), L³ is a 5-membered lactone.

In another embodiment of the compound of formula (III), L³ is a 5-membered lactam.

In yet another embodiment, preferred R¹—R³ L², and ring A groups are those presented above for formulae (I) and (II).

Yet another embodiment is a compound of one of formulae (IV)-(XII):

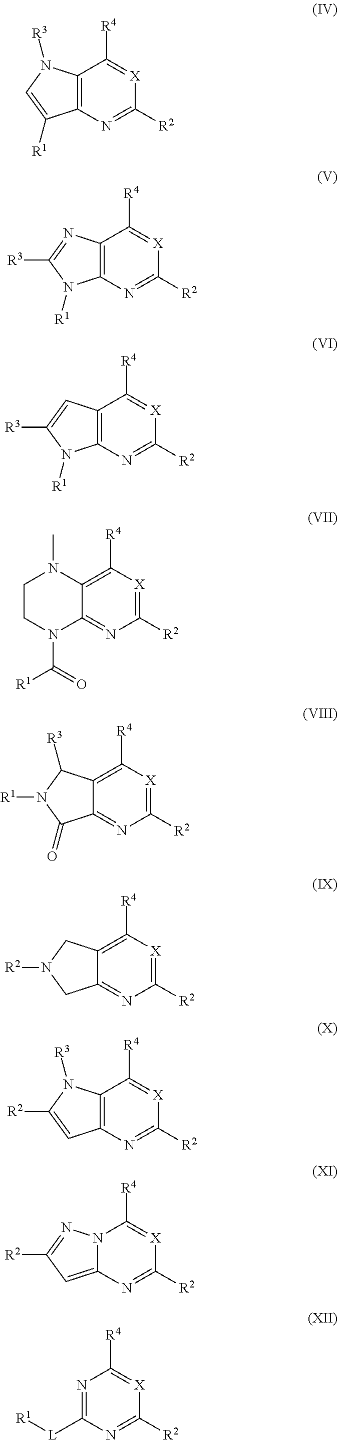

or a pharmaceutically acceptable salt thereof, wherein

R¹, R², and R³ are each independently hydrogen, hydroxy, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl;

R⁴ is a substituted or unsubstituted nitrogen- and/or oxygen-containing heterocyclyl;

X is N, CH, or CR^{a};

L is —CH(CH₃)—, —CH₂CH(CH₃)—, —CH(CH₃)CH₂—, —N(CH₃)—, —N(R^{c}—N(R^{c})₂)—, —N(CH₃)C(O)—, C(O)N(CH₃)—, —N(CH₃)C(O)(CR^{a}R^{b})ₘ—, C₁-C₂ alkylene, —NR^{c}—, —O—, —S—, —C(O)—, —NHC(O)—, —C(O)NH—, —NR^{c}C(O)—, —NR^{c}C(O)(CR^{a}R^{b})ₘ—, —O—(CR^{a}R^{b})ₘ—, —(CR^{a}R^{b})ₘ—, —NR^{c}—(CR^{a}R^{b})ₘ—, or —S—(CR^{a}R^{b})ₘ—;

m is 1-4;

R^{a} is hydrogen, hydroxy, hydroxy(C₁₋₄)alkyl, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl, halogen, nitro, —OR^{d}, —SR^{d}, —NR^{d}R^{e}, —C(O)R^{d}, —C(S)R^{d}, —OC(O)R^{d}, —SC(O)R^{d}, OC(S)R^{d}, SC(S)R^{d}, —NR^{c}C(O)R^{d}, —NR^{c}C(S)R^{d}, —SO₂R^{c}, —S(O)R^{c}, —NR^{c}SO₂R^{d}, —OS(O)₂R^{d}, —OP(O)R^{d}R^{e}, or —P(O)R^{d}R^{e}, where

R^{c} is a hydrogen or C₁₋₆ alkyl (e.g., C₁-C₄ alkyl), and

each occurrence of R^{d} and R^{e} are independently hydrogen, substituted or unsubstituted alkyl, substituted or unsubstituted alkenyl, substituted or unsubstituted alkynyl, substituted or unsubstituted alkoxy, substituted or unsubstituted aryl, substituted or unsubstituted heteroaryl, substituted or unsubstituted cycloalkyl, or substituted or unsubstituted heterocyclyl.

In preferred embodiments of the compounds of formulae (IV)-(XII), R¹ is selected from

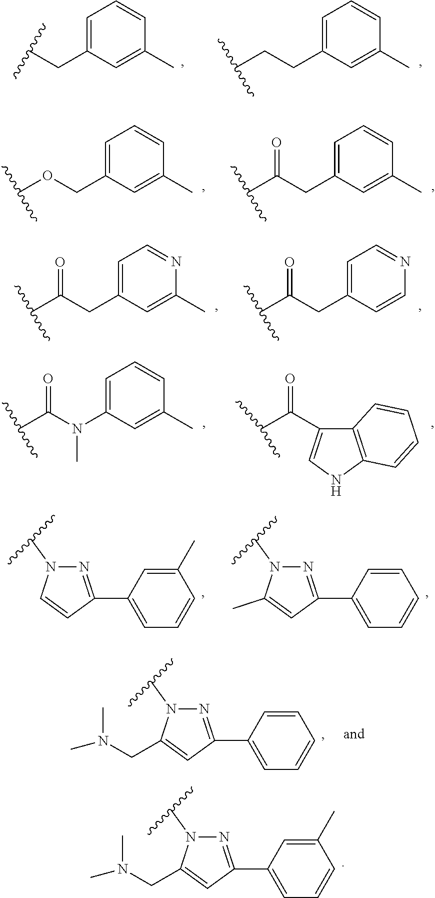

In preferred embodiments of the compounds of formulae (IV)-(XII), R² is selected from

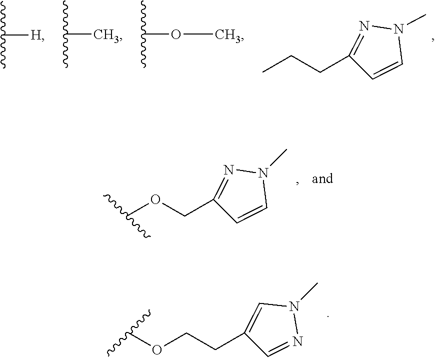

In preferred embodiments of the compounds of formulae (IV)-(XI), R³ is selected from

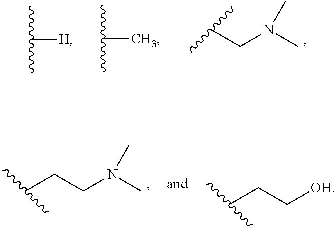

In some embodiments, R⁴ is a substituted or unsubstituted, saturated or unsaturated nitrogen- or oxygen-containing heterocyclyl. For instance, R⁴ can be a substituted or unsubstituted, saturated or unsaturated 5-10 membered (such as a 5-8 membered) mono- or bi-cyclic heterocyclyl having at least one nitrogen or oxygen ring atom. In some embodiments, R⁴ is a substituted or unsubstituted 5-10 membered (such as a 5-8 membered) mono- or bi-cyclic heterocyclyl having at least one nitrogen atom and optionally an oxygen ring atom, where the nitrogen ring atom is directly attached to the rest of the molecule (the bicyclic core shown in one of formulae (IV)-(XI)). In some instances, R⁴ is a substituted or unsubstituted (unsaturated) 5-membered monocyclic heterocyclyl having an oxygen ring atom or a nitrogen ring atom.

In some embodiments, R⁴ is a substituted or unsubstituted, saturated or unsaturated 6-membered monocyclic heterocyclyl having an oxygen ring atom and optionally a nitrogen ring atom. In yet another embodiment, R⁴ is a saturated 8-membered bicyclic heterocyclyl having a nitrogen ring atom and an oxygen ring atom. In some embodiments of the compounds of formulae (IV)-(XII), R⁴ is selected from

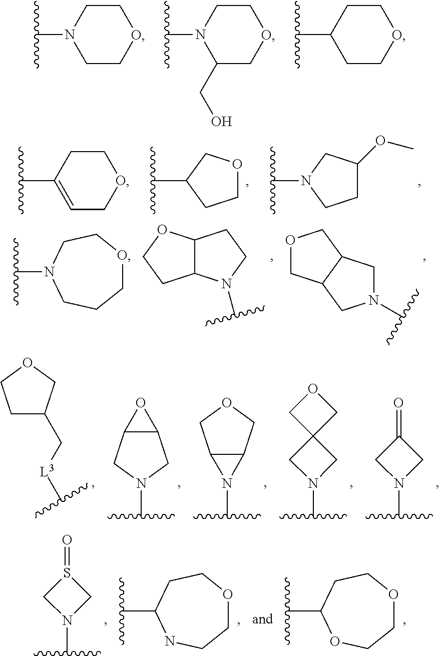

In preferred embodiments of the compounds of formulae (IV)-(XI), R⁴ is selected from

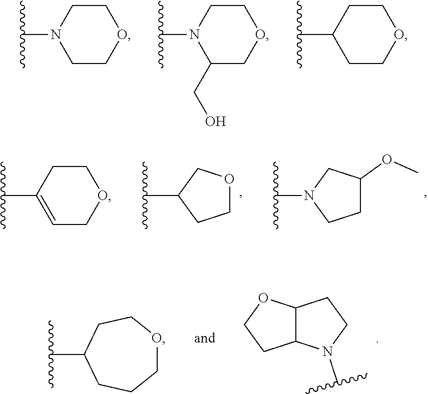

In another preferred embodiment, R⁴ is

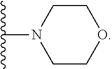 [SMILES: *N1CCOCC1]

In another preferred embodiment, R⁴ is

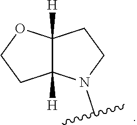 [SMILES: *N1CC[C@H]2OCC[C@H]21]

The variable L² in formulae II and III and ring A and L³ in formula III can be a 5-membered lactam. The 5-membered lactam in each of these positions can be selected from:

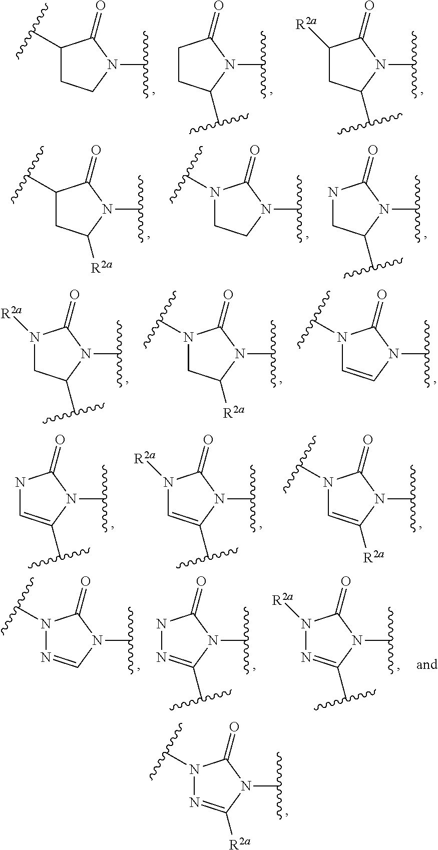

wherein

each squiggly line represents a point of attachment to adjacent groups (e.g., when the lactam is at position L² in formula II, one squiggly line represents a point of attachment to the R¹ group and the other squiggly line represents a point of attachment to the central pyrimidine ring), and

R^{2a} is selected from the R² groups listed above.

Exemplary compounds of the present include those listed below and pharmaceutically salts thereof.

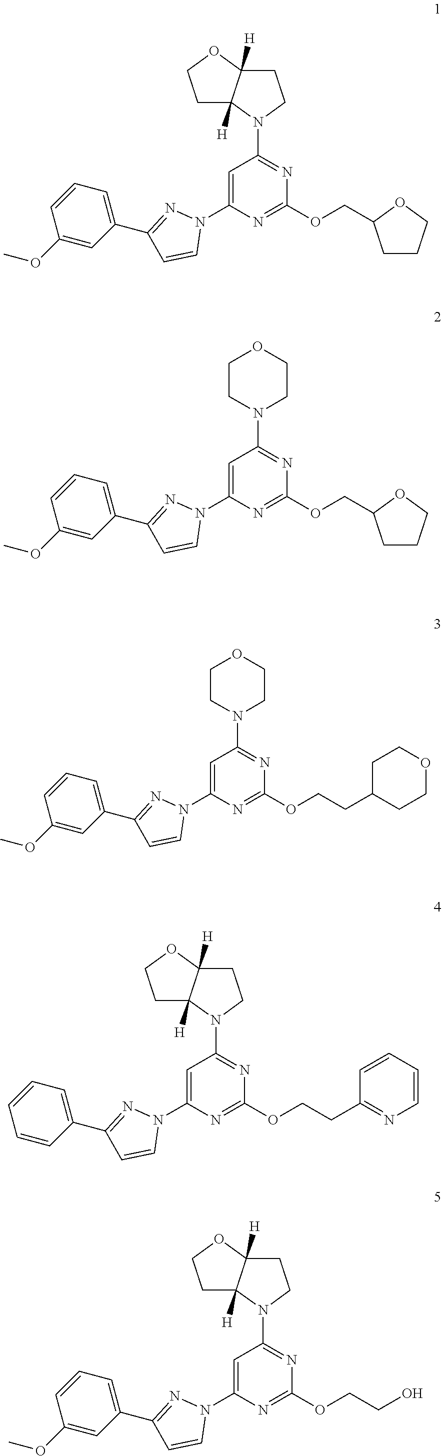 [SMILES: COc1cccc(-c2ccn(-c3cc(N4CC[C@H]5OCC[C@H]54)nc(OCC4CCCO4)n3)n2)c1 ; COc1cccc(-c2ccn(-c3cc(N4CCOCC4)nc(OCC4CCCO4)n3)n2)c1 ; COc1cccc(-c2ccn(-c3cc(N4CCOCC4)nc(OCCC4CCOCC4)n3)n2)c1 ; c1ccc(-c2ccn(-c3cc(N4CC[C@H]5OCC[C@H]54)nc(OCCc4ccccn4)n3)n2)cc1 ; COc1cccc(-c2ccn(-c3cc(N4CC[C@H]5OCC[C@H]54)nc(OCCO)n3)n2)c1]

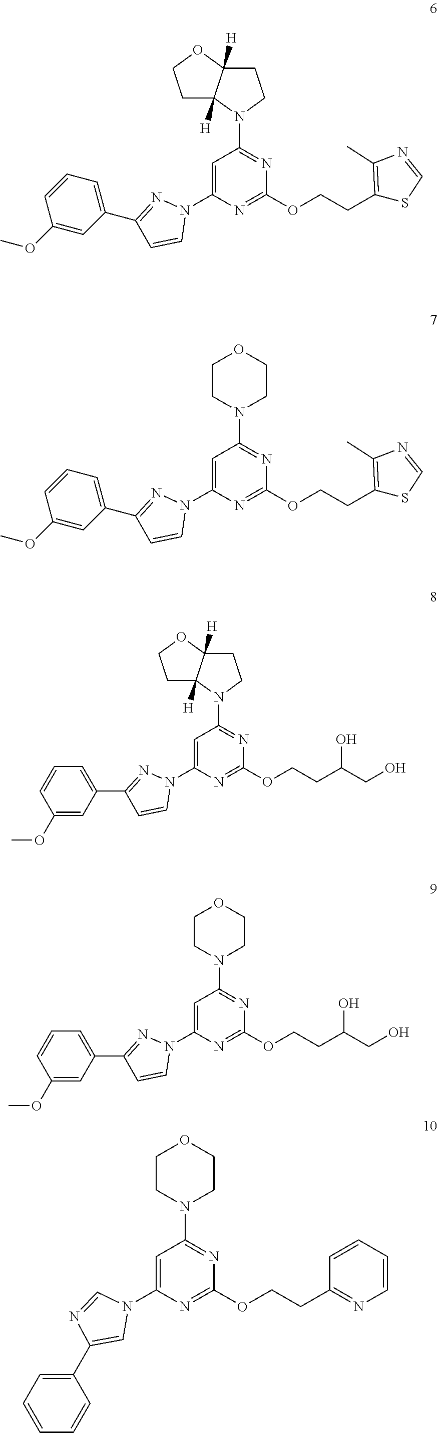 [SMILES: COc1cccc(-c2ccn(-c3cc(N4CC[C@H]5OCC[C@H]54)nc(OCCc4scnc4C)n3)n2)c1 ; COc1cccc(-c2ccn(-c3cc(N4CCOCC4)nc(OCCc4scnc4C)n3)n2)c1 ; COc1cccc(-c2ccn(-c3cc(N4CC[C@H]5OCC[C@H]54)nc(OCCC(O)CO)n3)n2)c1 ; COc1cccc(-c2ccn(-c3cc(N4CCOCC4)nc(OCCC(O)CO)n3)n2)c1 ; c1ccc(-c2cn(-c3cc(N4CCOCC4)nc(OCCc4ccccn4)n3)cn2)cc1]

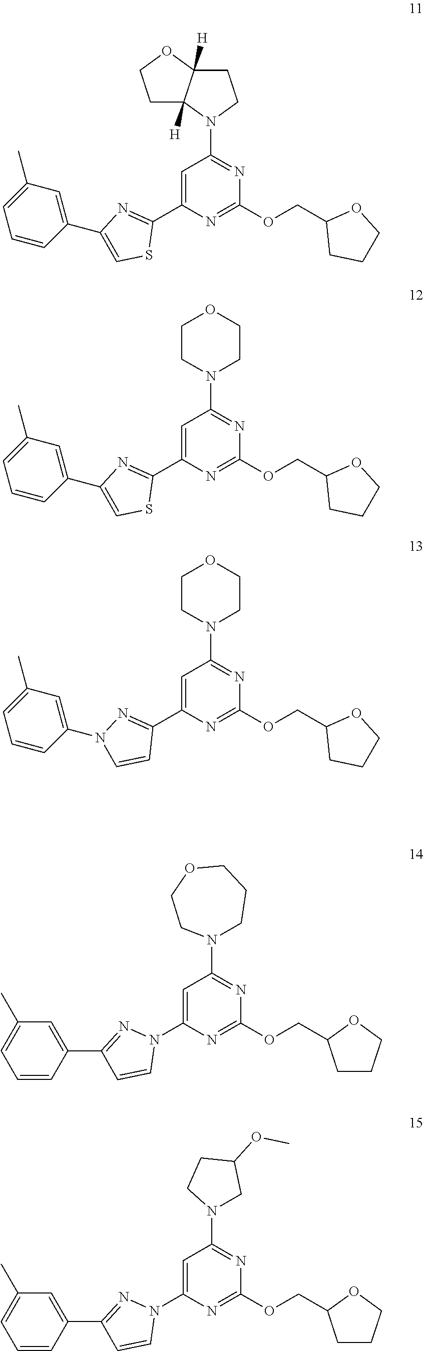 [SMILES: Cc1cccc(-c2csc(-c3cc(N4CC[C@H]5OCC[C@H]54)nc(OCC4CCCO4)n3)n2)c1 ; Cc1cccc(-c2csc(-c3cc(N4CCOCC4)nc(OCC4CCCO4)n3)n2)c1 ; Cc1cccc(-n2ccc(-c3cc(N4CCOCC4)nc(OCC4CCCO4)n3)n2)c1 ; Cc1cccc(-c2ccn(-c3cc(N4CCCOCC4)nc(OCC4CCCO4)n3)n2)c1 ; COC1CCN(c2cc(-n3ccc(-c4cccc(C)c4)n3)nc(OCC3CCCO3)n2)C1]

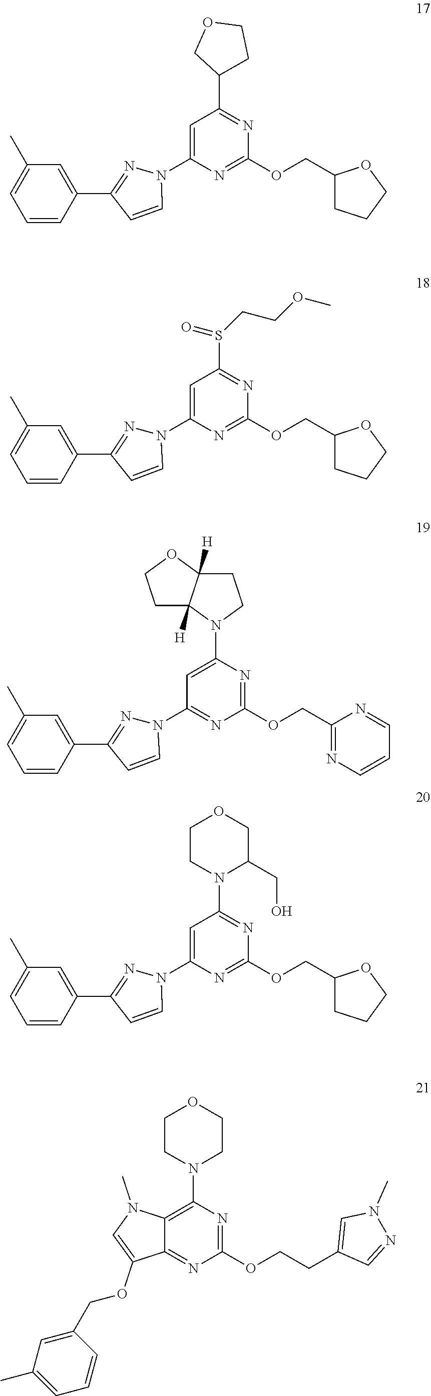 [SMILES: Cc1cccc(-c2ccn(-c3cc(C4CCOC4)nc(OCC4CCCO4)n3)n2)c1 ; COCCS(=O)c1cc(-n2ccc(-c3cccc(C)c3)n2)nc(OCC2CCCO2)n1 ; Cc1cccc(-c2ccn(-c3cc(N4CC[C@H]5OCC[C@H]54)nc(OCc4ncccn4)n3)n2)c1 ; Cc1cccc(-c2ccn(-c3cc(N4CCOCC4CO)nc(OCC4CCCO4)n3)n2)c1 ; Cc1cccc(COc2cn(C)c3c(N4CCOCC4)nc(OCCc4cnn(C)c4)nc23)c1]

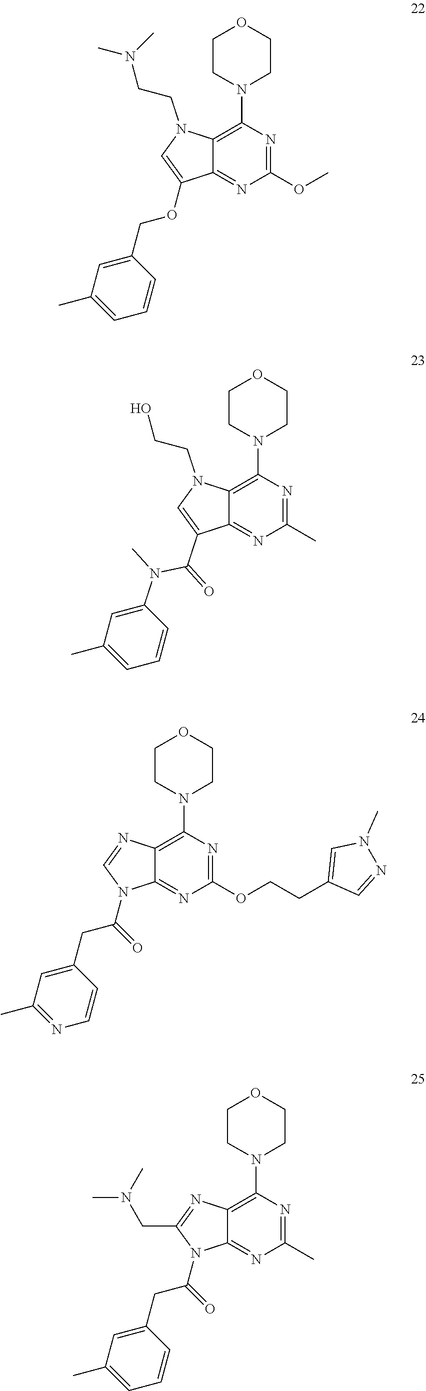

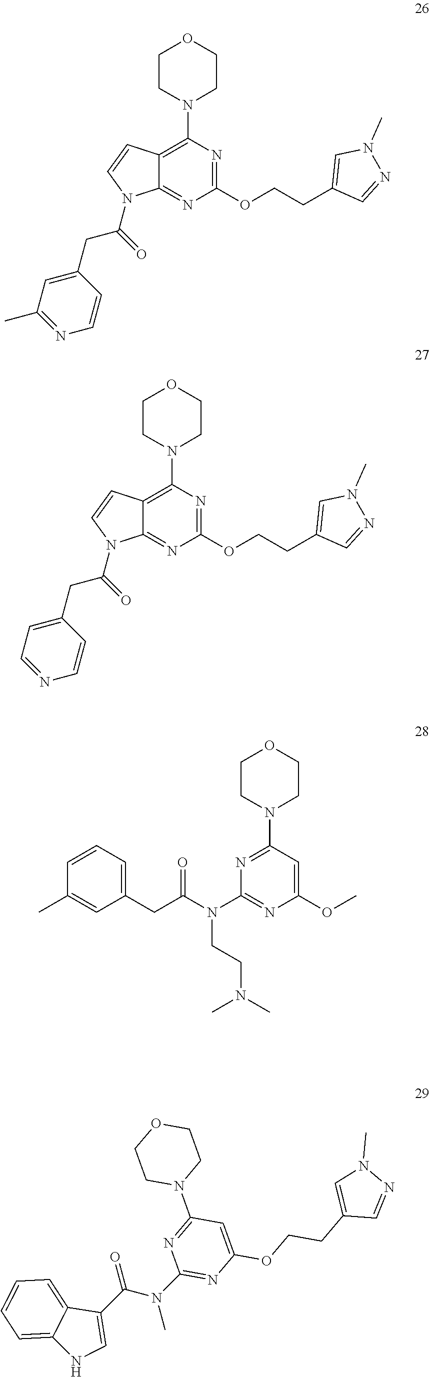

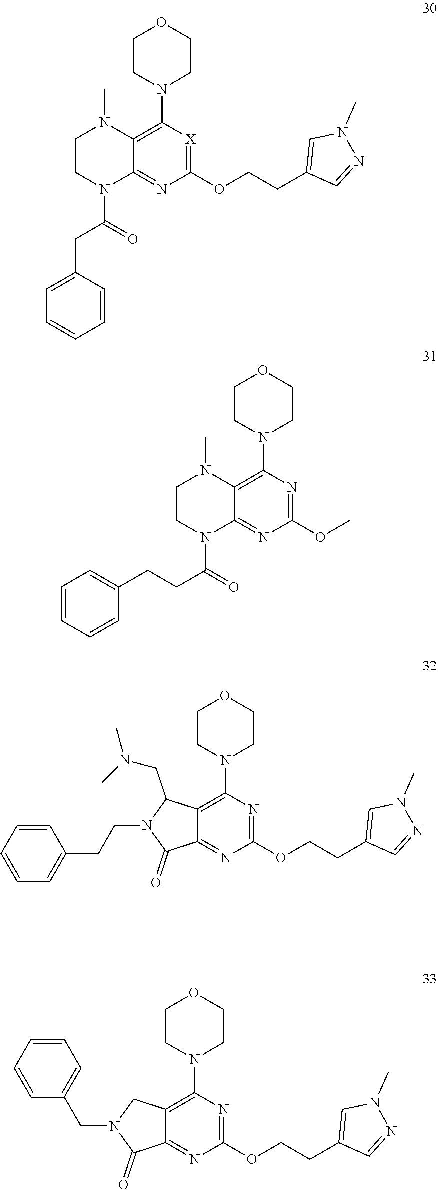

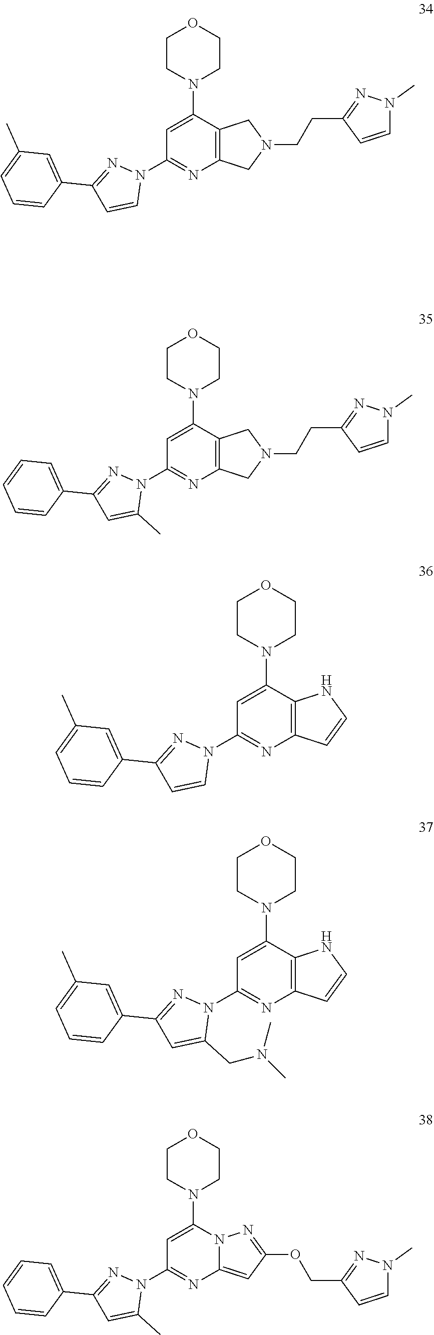 [SMILES: Cc1cccc(-c2ccn(-c3cc(N4CCOCC4)c4c(n3)CN(CCc3ccn(C)n3)C4)n2)c1 ; Cc1cc(-c2ccccc2)nn1-c1cc(N2CCOCC2)c2c(n1)CN(CCc1ccn(C)n1)C2 ; Cc1cccc(-c2ccn(-c3cc(N4CCOCC4)c4[nH]ccc4n3)n2)c1 ; Cc1cccc(-c2cc(CN(C)C)n(-c3cc(N4CCOCC4)c4[nH]ccc4n3)n2)c1 ; Cc1cc(-c2ccccc2)nn1-c1cc(N2CCOCC2)n2nc(OCc3ccn(C)n3)cc2n1]

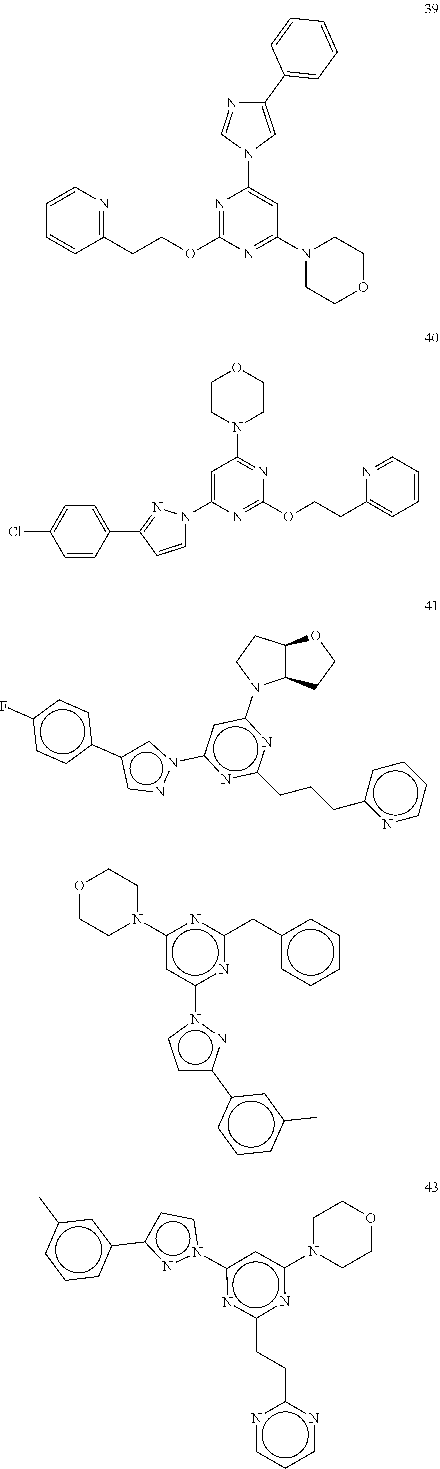

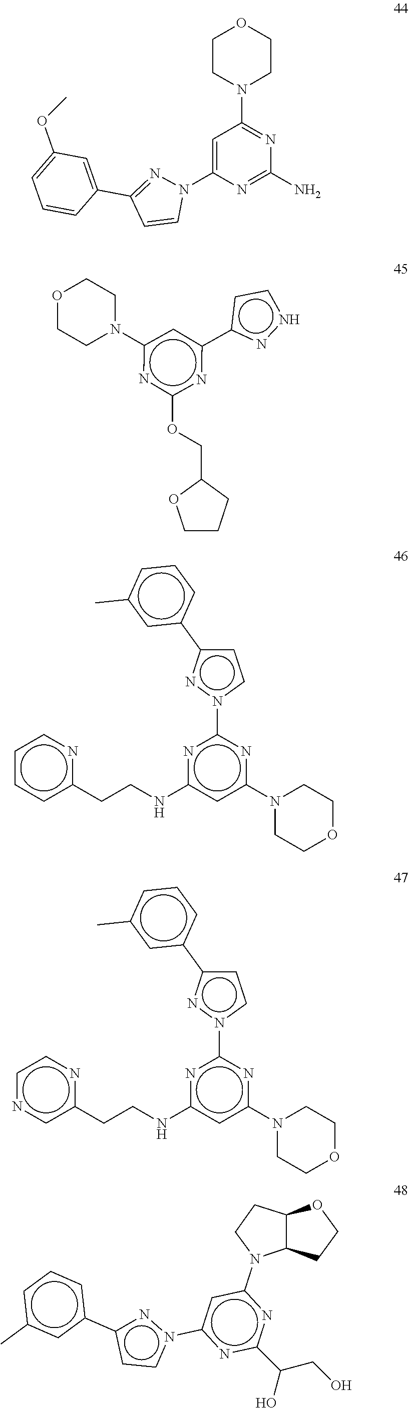

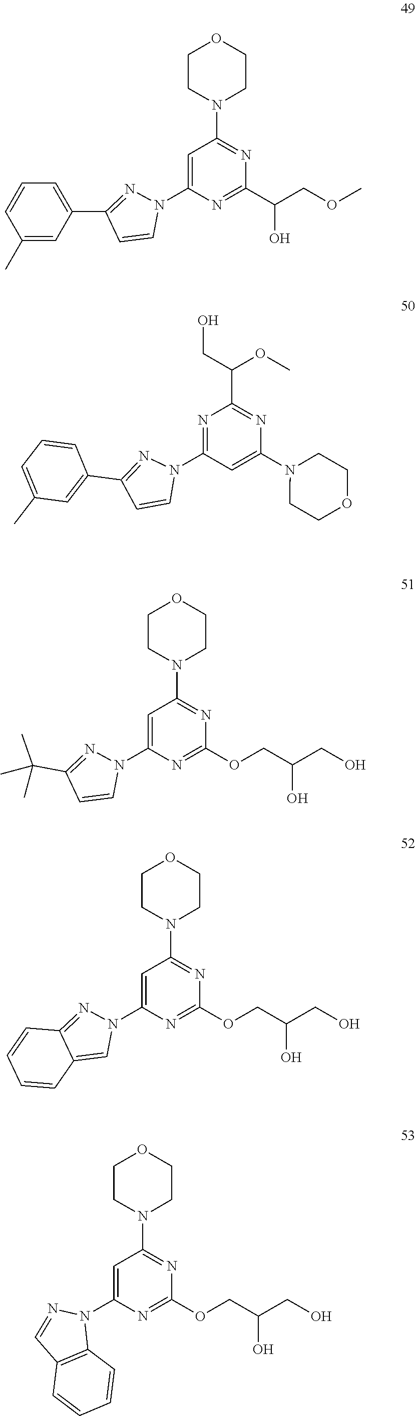

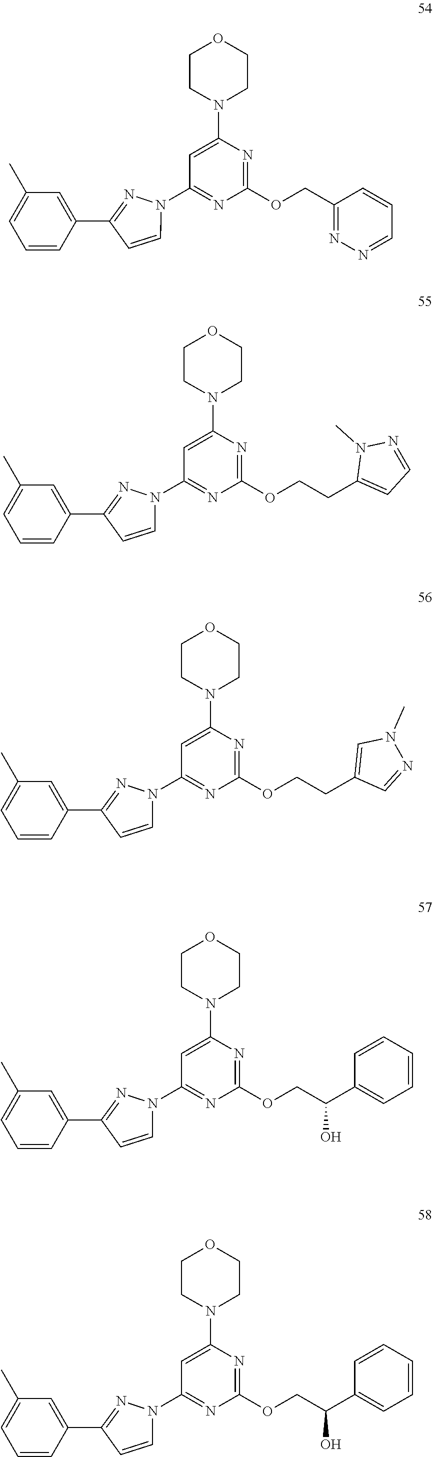 [SMILES: Cc1cccc(-c2ccn(-c3cc(N4CCOCC4)nc(OCc4cccnn4)n3)n2)c1 ; Cc1cccc(-c2ccn(-c3cc(N4CCOCC4)nc(OCCc4ccnn4C)n3)n2)c1 ; Cc1cccc(-c2ccn(-c3cc(N4CCOCC4)nc(OCCc4cnn(C)c4)n3)n2)c1 ; Cc1cccc(-c2ccn(-c3cc(N4CCOCC4)nc(OC[C@@H](O)c4ccccc4)n3)n2)c1 ; Cc1cccc(-c2ccn(-c3cc(N4CCOCC4)nc(OC[C@H](O)c4ccccc4)n3)n2)c1]

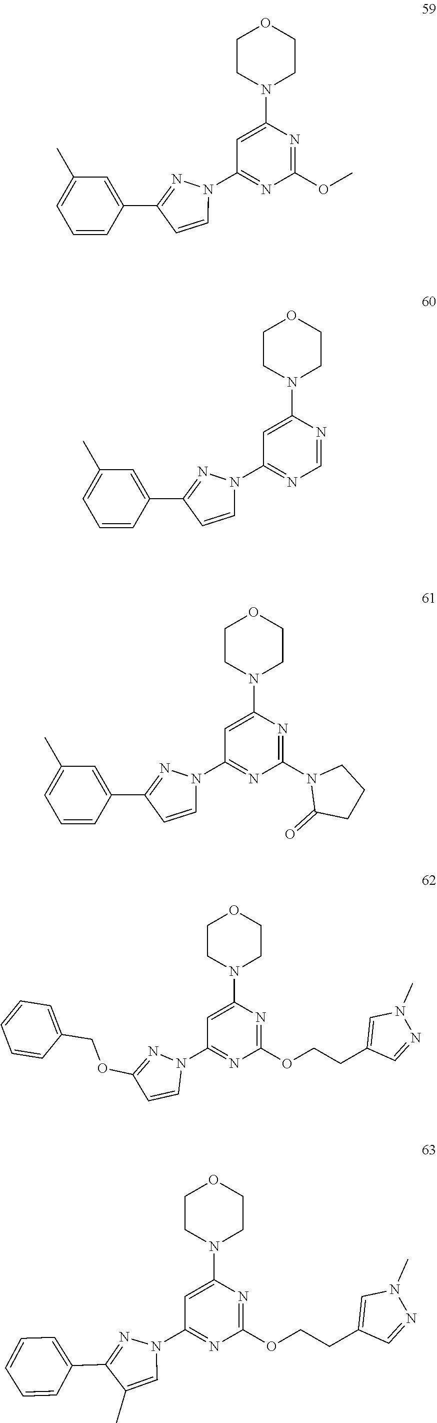 [SMILES: COc1nc(N2CCOCC2)cc(-n2ccc(-c3cccc(C)c3)n2)n1 ; Cc1cccc(-c2ccn(-c3cc(N4CCOCC4)ncn3)n2)c1 ; Cc1cccc(-c2ccn(-c3cc(N4CCOCC4)nc(N4CCCC4=O)n3)n2)c1 ; Cn1cc(CCOc2nc(N3CCOCC3)cc(-n3ccc(OCc4ccccc4)n3)n2)cn1 ; Cc1cn(-c2cc(N3CCOCC3)nc(OCCc3cnn(C)c3)n2)nc1-c1ccccc1]

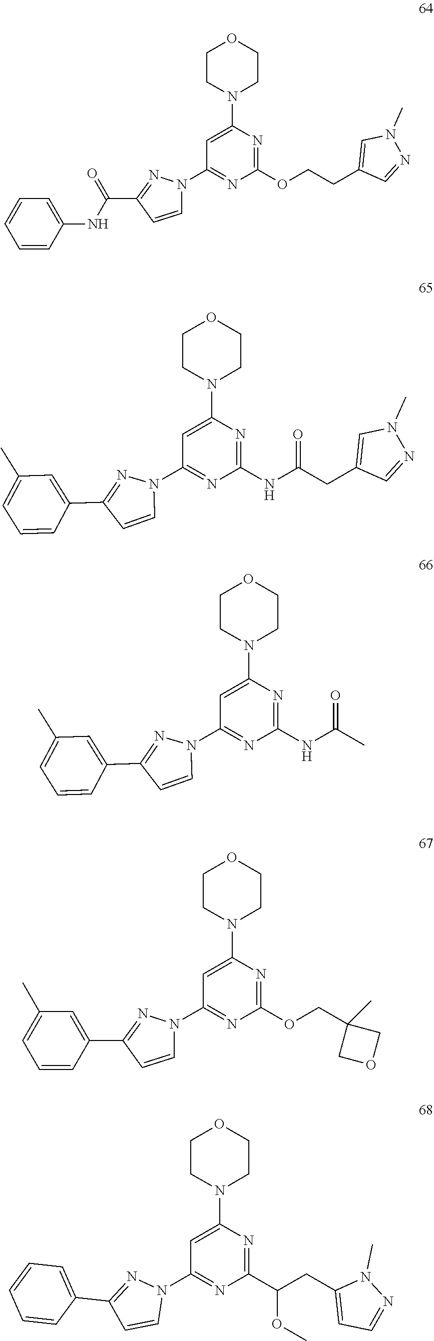 [SMILES: Cn1cc(CCOc2nc(N3CCOCC3)cc(-n3ccc(C(=O)Nc4ccccc4)n3)n2)cn1 ; Cc1cccc(-c2ccn(-c3cc(N4CCOCC4)nc(NC(=O)Cc4cnn(C)c4)n3)n2)c1 ; CC(=O)Nc1nc(N2CCOCC2)cc(-n2ccc(-c3cccc(C)c3)n2)n1 ; Cc1cccc(-c2ccn(-c3cc(N4CCOCC4)nc(OCC4(C)COC4)n3)n2)c1 ; COC(Cc1ccnn1C)c1nc(N2CCOCC2)cc(-n2ccc(-c3ccccc3)n2)n1]

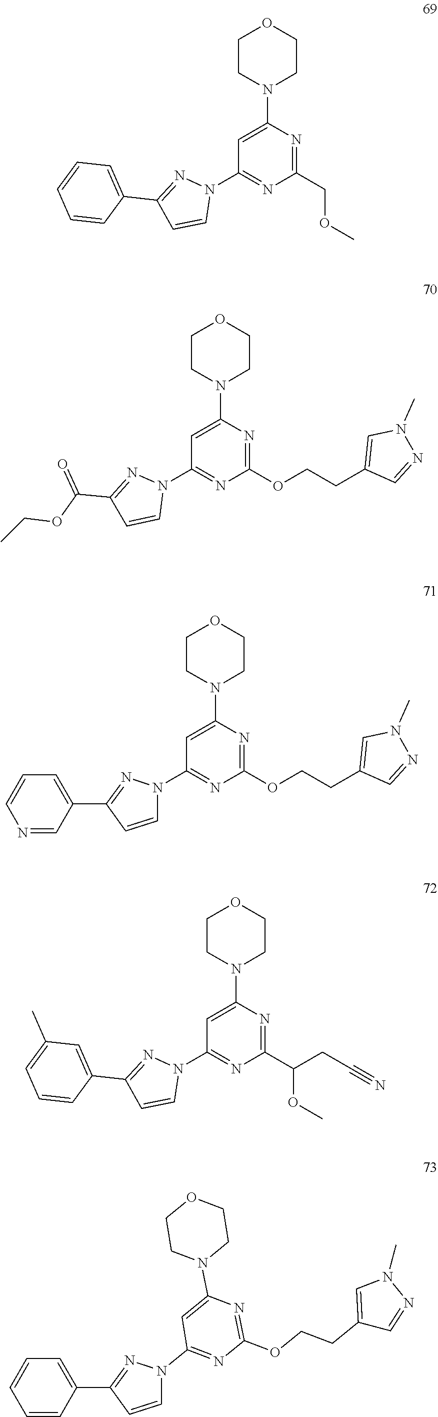

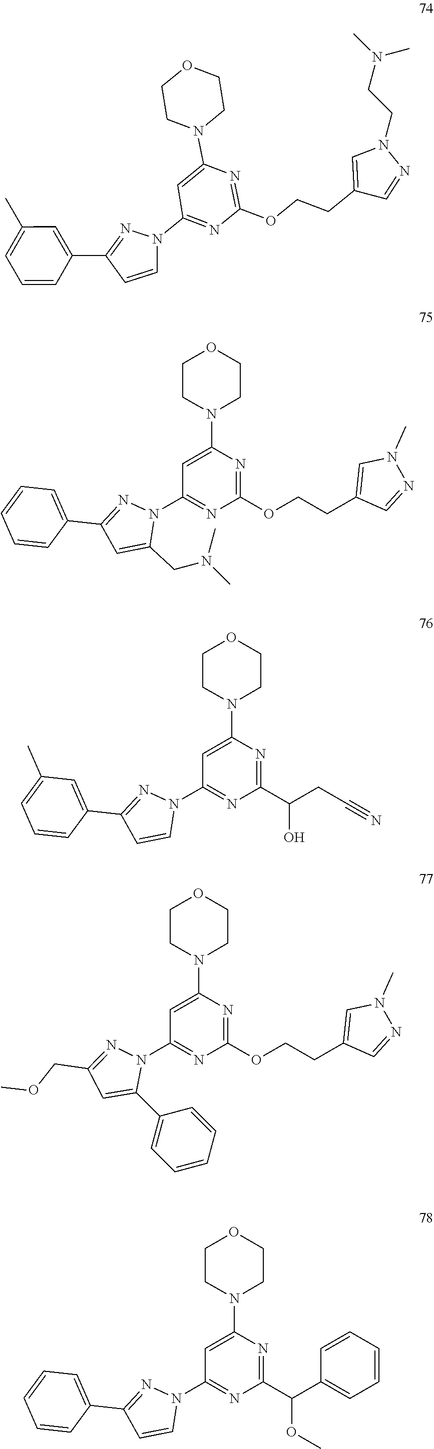

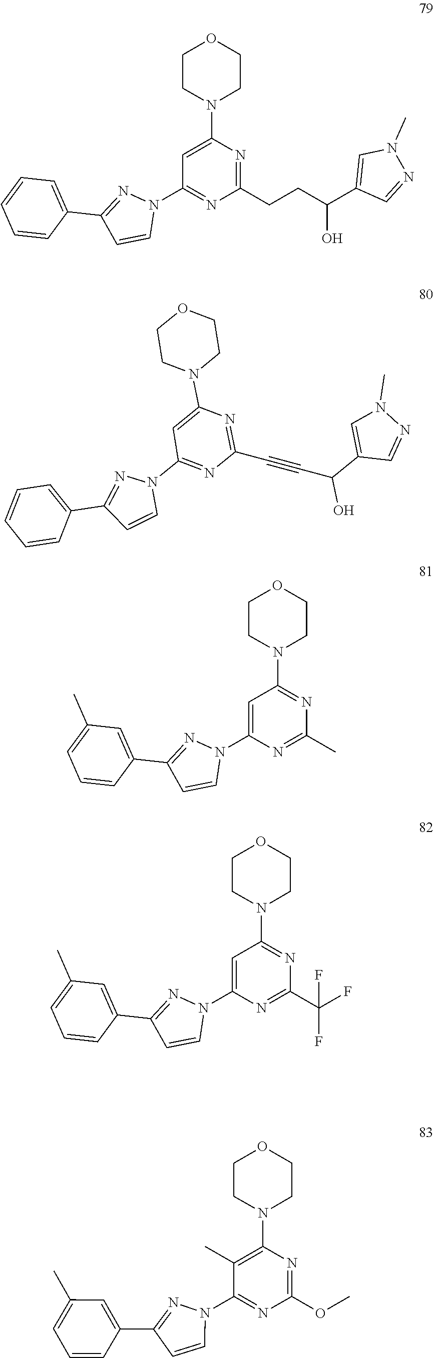 [SMILES: Cn1cc(C(O)CCc2nc(N3CCOCC3)cc(-n3ccc(-c4ccccc4)n3)n2)cn1 ; Cn1cc(C(O)C#Cc2nc(N3CCOCC3)cc(-n3ccc(-c4ccccc4)n3)n2)cn1 ; Cc1cccc(-c2ccn(-c3cc(N4CCOCC4)nc(C)n3)n2)c1 ; Cc1cccc(-c2ccn(-c3cc(N4CCOCC4)nc(C(F)(F)F)n3)n2)c1 ; COc1nc(N2CCOCC2)c(C)c(-n2ccc(-c3cccc(C)c3)n2)n1]

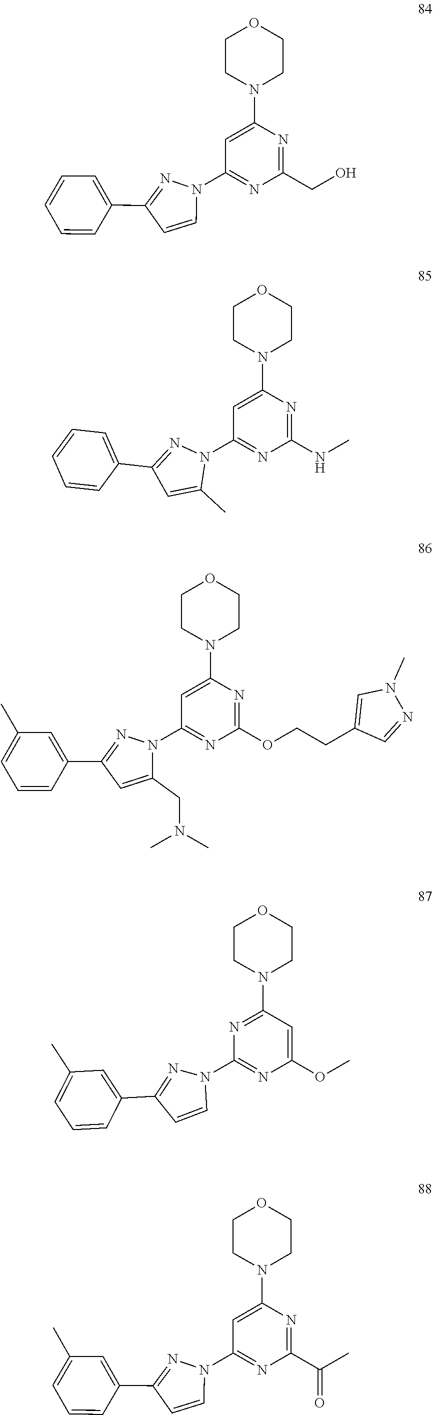

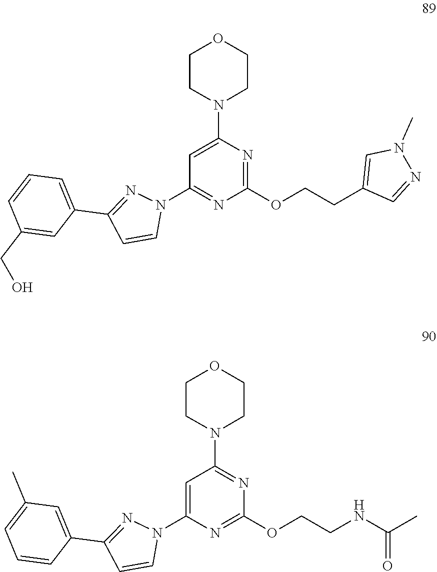 [SMILES: Cn1cc(CCOc2nc(N3CCOCC3)cc(-n3ccc(-c4cccc(CO)c4)n3)n2)cn1 ; CC(=O)NCCOc1nc(N2CCOCC2)cc(-n2ccc(-c3cccc(C)c3)n2)n1]

### Pharmaceutical Compositions

One embodiment is a pharmaceutical composition suitable for use in a subject, such as a human. The pharmaceutical composition may comprise at least one pharmaceutically acceptable excipient or carrier, in addition to the active ingredients (PIKfyve inhibitor and TMPRSS-2 inhibitor).

The pharmaceutical composition may also include at least one additional active agent, such as an alkylating agent, an intercalating agent, a tubulin binding agent, a corticosteroid, or any combination of any of the foregoing. Other additional active agents that can be included are apilimod, APY0201, YM-201636, remdesivir, favipiravir, and any combination of any of the foregoing.

The pharmaceutical composition may include one or more non-therapeutic agents, such as ondansetron, granisetron, dolasetron, palonosetron, pindolol, risperidone, or any combination of any of the foregoing.

A pharmaceutical composition can be provided as a dosage unit form, such as an ampoule, a vial, a suppository, a dragee, a tablet, or a capsule.

The pharmaceutical compositions can take any suitable form (e.g., liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration, in the form of a sterile aqueous solution or dispersion suitable for administration by either direct injection or by addition to sterile infusion fluids for intravenous infusion, or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

### Preparation of Certain PIKfyve Inhibitors

The compounds of the present invention may be prepared as follows.

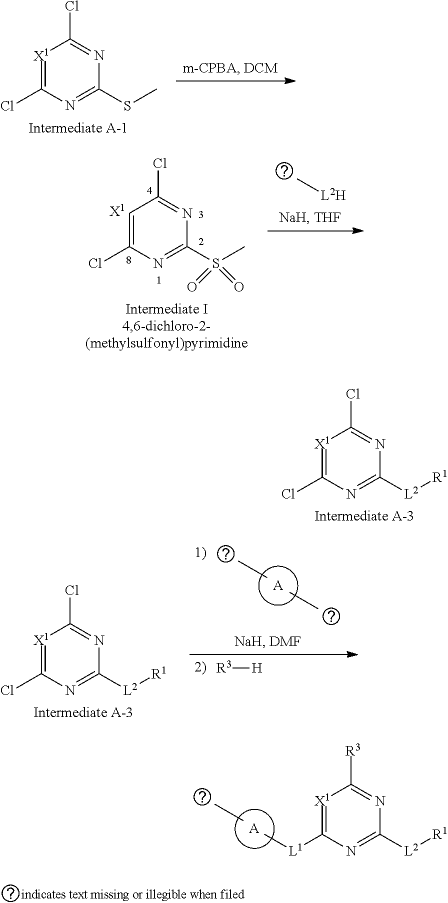

Starting intermediate A-1 is oxidized, for example by reaction with m-CPBA (meta-chloroperoxybenzoic acid) in a solvent, such as dichloromethane, to produce intermediate I. Intermediate I is then reacted with R¹-L²H, for example, in the presence of a base (such as NaH) and in a solvent, such as THF, to form Intermediate A-3. Intermediate A-3 is first reacted with

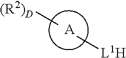

and then with R³—H to form the final compound.

### Examples

The examples are illustrative only and do not limit the claimed invention.

### Synthesis of 4,6-dichloro-2-(methylsulfonyl)pyrimidine (Intermediate I)

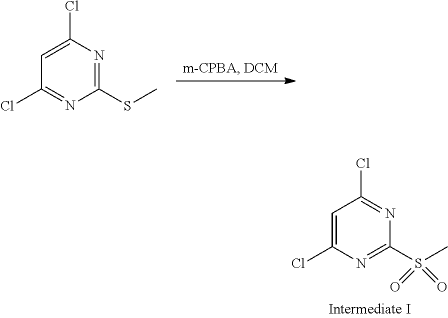

To a solution of 4,6-dichloro-2-(methylthio)pyrimidine (9.75 g, 50 mmol) in dichloromethane (DCM) was slowly added meta-chloroperoxybenzoic acid (mCPBA) 22.4 g, 130 mmol) at 0° C. The reaction was allowed to warm to room temperature (RT) and stirred overnight. The mixture was quenched with an aqueous solution of 1M NaOH, extracted with DCM, washed with sat. aq. NaHCO₃ as well as brine, and the organic phase dried (MgSO₄), filtered and evaporated to give Intermediate I, 4,6-dichloro-2-(methylsulfonyl)pyrimidine, as a white solid (11.39 g). The product was used crude.

LC/MS (mobile phase 5-100% ACN in 3 min), Rt=0.94 min, (M+H)⁺227

¹H NMR (300 MHz DMSO-d6): δ 8.30 (1H, s), 3.32 (3H, s)

### Synthesis of 4,6-dichloro-2-(2-pyridin-2-yl)ethoxy)pyrimidine (Intermediate II)

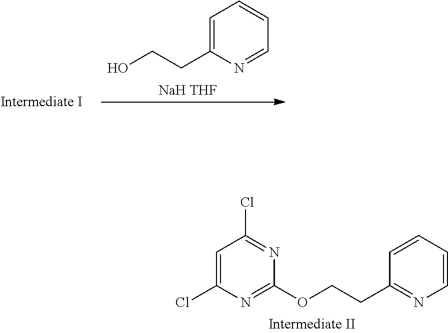 [SMILES: OCCc1ccccn1 ; Clc1cc(Cl)nc(OCCc2ccccn2)n1 ; "NaH THF"]

To a solution of Intermediate I (11.3 g, 50 mmol) in tetrahydrofuran (THF) (60 ml) was added NaH (2.9 g, 72.5 mmol). The temperature was lowered to −78° C. and 2-(pyridin-2-yl)ethan-1-ol (6.5 g, 52.5 mmol) in THF (60 ml) was added dropwise. The reaction was stirred for 1 h at −78° C., and worked up by addition of water, followed by extraction with ethyl acetate (EtOAc), dried (MgSO₄), filtered and evaporated. The crude product was purified by silica gel chromatography using a gradient of hexane: EtOAc 9:1 to hexane: EtOAc 7:3. After evaporation of the correct fractions, 6.7 g of Intermediate II, 4,6-dichloro-2-(2-pyridin-2-yl)ethoxy)pyrimidine was obtained as a white solid.

LC/MS (mobile phase 5-100% ACN in 3 min), Rt=1.68 min, (M+H)⁺270

¹H NMR (300 MHz DMSO-d6): δ 8.43 (1H, d), 7.66 (2H, m), 7.22 (1H, d), 7.15 (1H, m), 4.21 (2H, m), 3.29 (2H, m)

### Synthesis of (3aR,6aR)-hexahydro-2H-furo[3,2-b]pyrrole (Intermediate III)

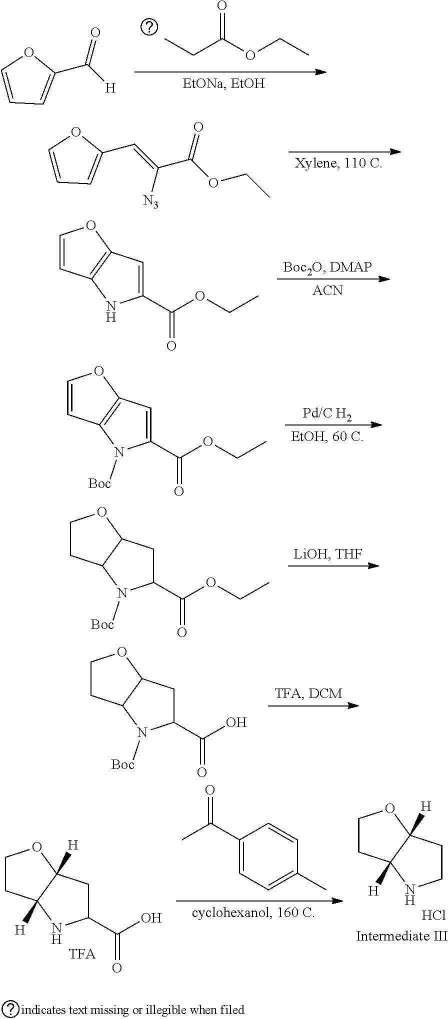

To a solution of furan-2-carbaldehyde and ethyl 2-azidopropanoate in etanol (EtOH) was added sodium ethoxide (1.1 eq) and the reaction heated at reflux overnight. Evaporated, redissolved in ethyl acetate, and washed with sat. aq. sodium bicarbonate, the organic phase dried (MgSO₄), filtered and evaporated. The crude product was redissolved in xylene and heated at 160° C. for 4 hours, then evaporated and purified by silica gel chromatography (EtOAc/hexane). Re-dissolved in ACN, 4-Dimethylaminopyridine (DMAP) added (0.1 eq) and Boc₂O and stirred at rt for 5 h, worked up (as before), redissolved in EtOH and hydrogenated at 40 psi with Pd/C 105 for 2 h. Evaporated and treated with sat. aq. LiOH in THF at RT for 2 h. Evaporated and purified by LC/MS. The solid was redissolved in TFA/DCM 1:2 and stirred and rt for 2 h, evaporated, decarboxylated by heating, purified by LC/MS and covered to the HCl salt.

¹H NMR (300 MHz DMSO-d6): δ 83.68 (3H, m), 2.79 (3H, m), 2.01 (3H, m), 1.76 (2H, m)

### Synthesis of 4-chloro-6-(3-(3-methoxyphenyl)-1H-pyrazol-1-yl)-2-(2-(pyridin-2-yl)ethoxy)pyrimidine (Intermediate IV)

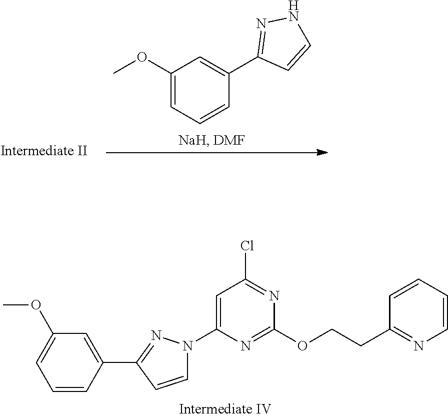 [SMILES: COc1cccc(-c2cc[nH]n2)c1 ; COc1cccc(-c2ccn(-c3cc(Cl)nc(OCCc4ccccn4)n3)n2)c1]

To a solution of Intermediate II, 4,6-dichloro-2-(2-(pyridin-2-yl)ethoxy)pyrimidine (269 mg, 1 mmol) in DMF (10 ml) was added 3-(3-methoxyphenyl)-1H-pyrazole (191 mg, 1.1 mmol) and NaH (19 mg, 1.2 mmol). The reaction was stirred at RT overnight, quenched with water, extracted with EtOAc, dried (MgSO₄), filtered, evaporated and purified by LCMS to give 4-chloro-6-(3-phenyl-1H-pyrazol-1-yl)-2-(2-(pyridin-2-yl)ethoxy)pyrimidine (Intermediate IV) (188 mg).

LC/MS (M+H)⁺408

¹H NMR (300 MHz DMSO-d6): δ 8.43 (1H, m), 8.09 (1H, m), 7.76 (1H, s), 7.72 (2H, m), 7.47 (1H, m), 7.39 (1H, s), 7.18 (3H, m), 6.95 (1H, m), 4.25 (2H, m), 3.81 (3H, S), 3.30 (2H, m),

### Synthesis of (3aR,6aR)-4-(6-chloro-2-((tetrahydrofuran-2-yl)methoxy)pyrimidin-4-yl)hexahydro-2H-furo[3,2-b]pyrrole (Intermediate V)

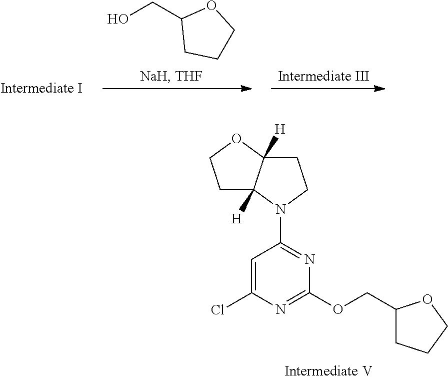 [SMILES: OCC1CCCO1 ; Clc1cc(N2CC[C@H]3OCC[C@H]32)nc(OCC2CCCO2)n1]

To 2.8 g of Intermediate I (12.3 mml) was added NaH (770 mg, 32 mmol) and tetrahydrofuran-2-yl)methanol (1.81 g, 16 mmol) at 0° C. in THF (200 ml). The reaction was stirred at 2 h at RT, and Intermediate III (1.81 g, 16 mmol) added, and the reaction stirred overnight at RT. The reaction was worked up by quenching with water, evaporation, re-dissolved in ethyl acetate, washed (saturated aq. sodium bicarbonate), dried (MgSO₄), filtered and evaporated. The crude mixture was purified by silica gel chromatography (hexane/ethyl acetate) to give 2.2 g of Intermediate V.

LC/MS (mobile phase 5-100% ACN in 5 min), Rt=3.36 min, (M+H)⁺326

¹H NMR (300 MHz CDCl₃): δ 7.29 (s, 1H), 4.49 (m, 5H), 3.37 (m, 6H), 2.24 (m, 2H), 2.13 (m, 1H), 1.90 (m, 5H)

### Synthesis of 4-(6-chloro-2-((tetrahydrofuran-2-yl)methoxy)pyrimidin-4-yl)morpholine (Intermediate VI)

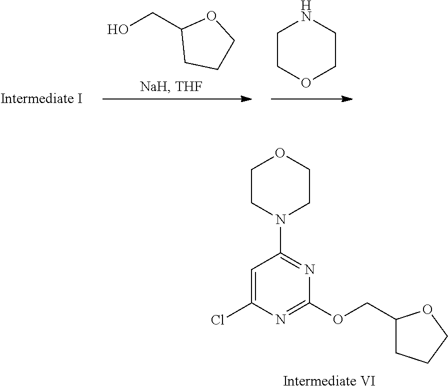

Intermediate VI was prepared by a method analogous to Intermediate V, except morpholine was added instead of Intermediate III.

LC/MS (mobile phase 5-100% ACN in 4 min), Rt=2.22 min, (M+H)⁺300

¹H NMR (300 MHz DMSO-d6): δ 6.62 (s, 1H), 4.19 (m, 1H), 4.14 (m, 1H), 3.77 (m, 1H), 3.62 (m, 9H), 1.97 (m, 1H), 1.85 (m, 2H), 1.62 (m, 1H)

### Synthesis of 4-chloro-2-((tetrahydrofuran-2-yl)methoxy)-6-(3-(m-tolyl)-1H-pyrazol-1-yl)pyrimidine (Intermediate VII)

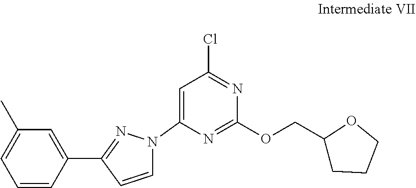 [SMILES: Cc1cccc(-c2ccn(-c3cc(Cl)nc(OCC4CCCO4)n3)n2)c1]

Intermediate VII was prepared by a method analogous to Intermediate IV, except tetrahydrofuran-2-yl)methanol was added in place of 2-(pyridin-2-yl)ethan-1-ol to generate the equivalent of Intermediate II and 5-(m-tolyl)-1H-pyrazole was added in place of 3-(3-methoxyphenyl)-1H-pyrazole to generate Intermediate VII.

LC/MS (mobile phase 5-100% ACN in 4 min), Rt=3.52 min, (M+H)⁺371

¹H NMR (300 MHz DMSO-d6): δ 8.75 (s, 1H), 7.77 (s, 1H), 7.81 (d, 1H), 7.68 (s, 1H), 7.39 (m, 1H), 7.27 (m, 1H), 7.21 (s, 1H), 4.40 (m, 2H), 2.16 (m, 1H), 3.83 (m, 1H, 3.72 (m, 1H), 2.40 (s, 3H), 2.06 (m, 1H), 1.88 (m, 2H), 1.72 (m, 1H)

### Synthesis of 4-chloro-2-(2-(tetrahydrofuran-2-yl)ethyl)-6-(3-(m-tolyl)-1H-pyrazol-1-yl)pyrimidine (Intermediate VIII)

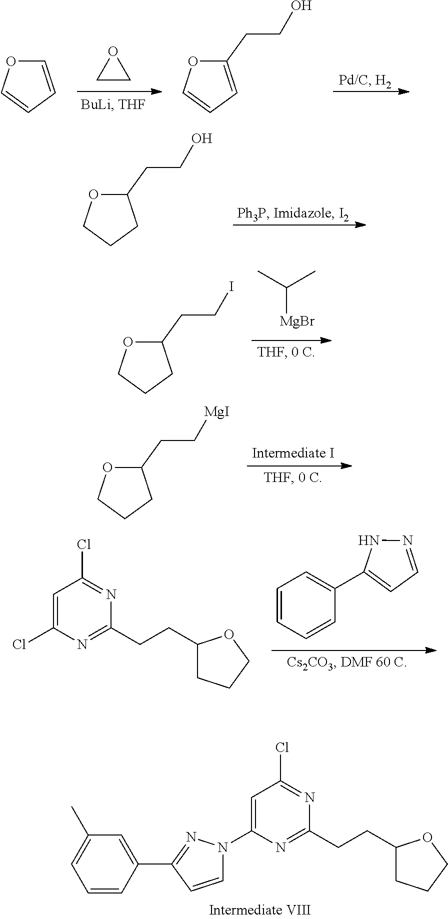

Intermediate VIII was prepared by the scheme shown above.

L/MS (mobile phase 5-100% ACN in 5 min), Rt=4.32 min, (M+H)⁺ 369

¹H NMR (300 MHz DMSO-d6): δ 8.72 (s, 1H), 7.89 (m, 2H), 7.81 (m, 1H), 7.39 (m, 1H), 7.26 (m, 1H), 7.20 (s, 1H), 3.82 (m, 1H), 3.75 (m, 1H), 3.60 (m, 1H), 2.90 (m, 2H), 2.40 (s, 3H), 2.00 (m, 3H), 1.82 (m, 2H), 1.49 (m, 1H)

### Example 1: Synthesis of 3aR,6aR)-4-(6-(3-(3-methoxyphenyl)-1H-pyrazol-1-yl)-2-((tetrahydrofuran-2-yl)methoxy)pyrimidin-4-yl)hexahydro-2H-furo[3,2-b]pyrrole (Compound 1)

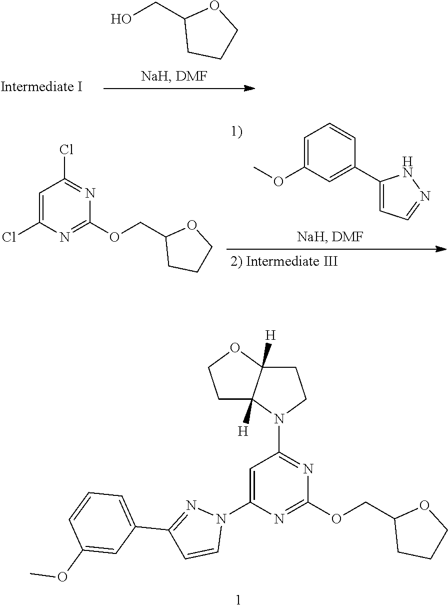 [SMILES: OCC1CCCO1 ; Clc1cc(Cl)nc(OCC2CCCO2)n1 ; COc1cccc(-c2ccn[nH]2)c1 ; COc1cccc(-c2ccn(-c3cc(N4CC[C@H]5OCC[C@H]54)nc(OCC4CCCO4)n3)n2)c1]

To a solution of Intermediate I, 4,6-dichloro-2-(methylsulfonyl)pyrimidine (113 mg, 0.5 mmol) in THF (5 ml), was added NaH (14 mg, 0.64 mmol) and the solution cooled to −78° C. Tetrahydrofuran-2-yl)methanol (51 mg, 0.48 mmol) was added dropwise as a solution in THF (1 ml), and the solution stirred for 1 h at −78° C., then quenched with water, extracted with EtOAc, dried (MgSO₄), filtered, evaporated and purified by silica gel chromatography (hexane/EtOAc) to give 16 mg of 4,6-dichloro-2-((tetrahydrofuran-2-yl)methoxy)pyrimidine. This was dissolved in dimethylformamide (DMF) (1 ml), NaH (4 mg, 0.18 mmol) was added, followed by 5-(3-methoxyphenyl)-1H-pyrazole (16 mg, 0.09 mmol), and the reaction mixture stirred for 1 h at RT. Intermediate III (44 mg) was added, and the reaction stirred overnight at RT, then quenched with water, extracted with EtOAc, dried (MgSO₄), filtered, evaporated and purified by LC/MS to give 8 mg of (3aR,6aR)-4-(6-(3-(3-methoxyphenyl)-1H-pyrazol-1-yl)-2-((tetrahydrofuran-2-yl)methoxy)pyrimidin-4-yl)hexahydro-2H-furo[3,2-b]pyrrole Compound 1.

LC/MS (mobile phase 5-100% ACN in 5 min), Rt=3.91 min, (M+H)⁺464

### Example 2: Synthesis of 4-(6-(3-(3-methoxyphenyl)-1H-pyrazol-1-yl)-2-((tetrahydrofuran-2-yl)methoxy)pyrimidin-4-yl)morpholine (Compound 2)

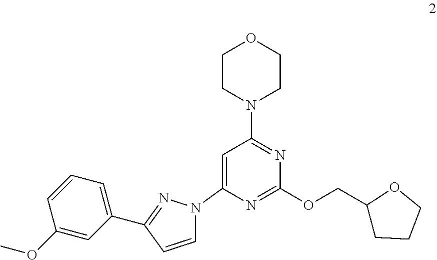 [SMILES: COc1cccc(-c2ccn(-c3cc(N4CCOCC4)nc(OCC4CCCO4)n3)n2)c1]

To a solution of Intermediate I, 4,6-dichloro-2-(methylsulfonyl)pyrimidine (113 mg, 0.5 mmol) in THF (5 ml), was added NaH (14 mg, 0.64 mmol) and the solution cooled to −78° C. Tetrahydrofuran-2-yl)methanol (51 mg, 0.48 mmol) was added dropwise as a solution in THF (1 ml), and the solution stirred for 1 h at −78° C., then quenched with water, extracted with EtOAc, dried (MgSO₄), filtered, evaporated and purified by silica gel chromatography (hexane/EtOAc) to give 23 mg of 4,6-dichloro-2-((tetrahydrofuran-2-yl)methoxy)pyrimidine. This was dissolved in DMF (3 ml), NaH (6 mg) was added, followed by 5-(3-methoxyphenyl)-1H-pyrazole (16 mg) and the reaction mixture stirred for 1 h at RT. Morpholine (9 ul) was added, and the reaction stirred overnight at rt, then quenched with water, extracted with EtOAc, dried (MgSO₄), filtered, evaporated and purified by LC/MS to give 6 mg of 4-(6-(3-(3-methoxyphenyl)-1H-pyrazol-1-yl)-2-((tetrahydrofuran-2-yl)methoxy)pyrimidin-4-yl)morpholine, Compound 2.

LC/MS (mobile phase 5-100% ACN in 5 min), Rt=3.90 min, (M+H)⁺438

### Example 3: Synthesis of 4-(6-(3-phenyl-1H-pyrazol-1-yl)-2-(2-(tetrahydro-2H-pyran-4-yl)ethoxy)pyrimidin-4-yl)morpholine (Compound 3)

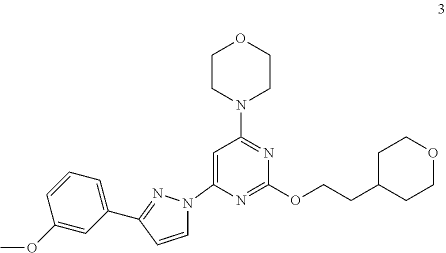 [SMILES: COc1cccc(-c2ccn(-c3cc(N4CCOCC4)nc(OCCC4CCOCC4)n3)n2)c1]

To a solution of Intermediate I, 4,6-dichloro-2-(methylsulfonyl)pyrimidine (113 mg, 0.5 mmol) in THF (5 ml), was added NaH (14 mg, 0.64 mmol) and the solution cooled to −78° C. 2-(tetrahydro-2H-pyran-4-yl)ethan-1-ol (65 mg) was added dropwise as a solution in THF (1 ml), and the solution stirred for 1 h at −78° C., then quenched with water, extracted with EtOAc, dried (MgSO₄), filtered, evaporated and purified by silica gel chromatography (hexane/EtOAc) to give 65 mg of 4-(2-(3,5-dichlorophenoxy)ethyl)tetrahydro-2H-pyran. This was dissolved in DMF (3 ml), NaH (9 mg) was added, followed by 5-(3-methoxyphenyl)-1H-pyrazole (41 mg) and the reaction mixture stirred for 1 h at RT. Morpholine (21 ul) was added, and the reaction stirred overnight at rt, then quenched with water, extracted with EtOAc, dried (MgSO₄), filtered, evaporated and purified by LC/MS to give 9 mg of 4-(6-(3-(3-methoxyphenyl)-1H-pyrazol-1-yl)-2-(2-(tetrahydro-2H-pyran-4-yl)ethoxy)pyrimidin-4-yl)morpholine, Compound 3.

LC/MS (mobile phase 5-100% ACN in 5 min), Rt=4.14 min, (M+H)⁺466

### Example 4: Synthesis of (3aR,6aR)-4-(6-(3-phenyl-1H-pyrazol-1-yl)-2-(2-(pyridin-2-yl)ethoxy)pyrimidin-4-yl)hexahydro-2H-furo[3,2-b]pyrrole (Compound 4)

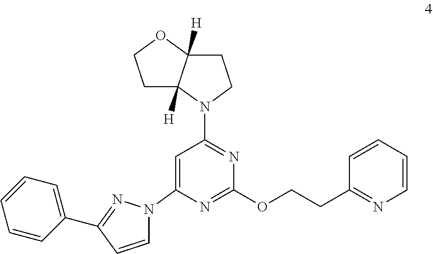 [SMILES: c1ccc(-c2ccn(-c3cc(N4CC[C@H]5OCC[C@H]54)nc(OCCc4ccccn4)n3)n2)cc1]

Compound 4 was prepared by a method analogous to that for Compound 1, except Intermediate III was used in place of morpholine and 5-phenyl-1H-pyrazole was used in place of 5-(3-methoxyphenyl)-1H-pyrazole.

LC/MS (mobile phase 5-100% ACN in 5 min), Rt=3.16 min, (M+H)⁺455

### Example 5: Synthesis of 2-((4-((3aR,6aR)-hexahydro-4H-furo[3,2-b]pyrrol-4-yl)-6-(3-(3-methoxyphenyl)-1H-pyrazol-1-yl)pyrimidin-2-yl)oxy)ethan-1-ol (Compound 5)

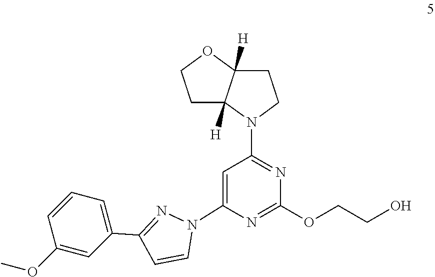 [SMILES: COc1cccc(-c2ccn(-c3cc(N4CC[C@H]5OCC[C@H]54)nc(OCCO)n3)n2)c1]

Compound 5 was prepared by a method analogous to that for Compound 1, except 2-(tert-butoxy)ethan-1-ol was added instead of tetrahydrofuran-2-yl)methanol, and the end product was treated with trifluoroacetic acid (TFA)/DCM 1:2 for 1 h at RT and evaporated prior to purification by LC/MS.

LC/MS (mobile phase 5-100% ACN in 5 min), Rt=3.16 min, (M+H)⁺424

### Example 6: Synthesis of (3aR,6aR)-4-(6-(3-(3-methoxyphenyl)-1H-pyrazol-1-yl)-2-(2-(4-methylthiazol-5-yl)ethoxy)pyrimidin-4-yl)hexahydro-2H-furo[3,2-b]pyrrole (Compound 6)

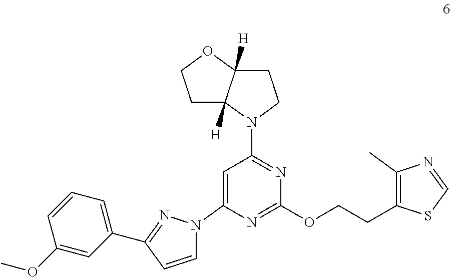 [SMILES: COc1cccc(-c2ccn(-c3cc(N4CC[C@H]5OCC[C@H]54)nc(OCCc4scnc4C)n3)n2)c1]

Compound 6 was prepared by a method analogous to that for Compound 1, except 2-(4-methylthiazol-5-yl)ethan-1-ol was added instead of tetrahydrofuran-2-yl)methanol.

LC/MS (mobile phase 5-100% ACN in 5 min), Rt=3.60 min, (M+H)⁺505

### Example 7: Synthesis of 4-(6-(3-(3-methoxyphenyl)-1H-pyrazol-1-yl)-2-(2-(4-methylthiazol-5-yl)ethoxy)pyrimidin-4-yl)morpholine (Compound 7)

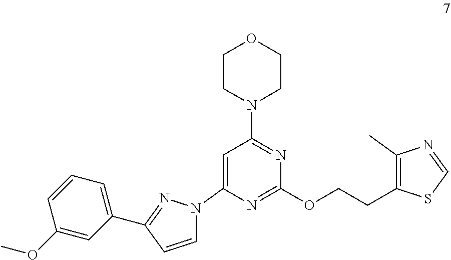 [SMILES: COc1cccc(-c2ccn(-c3cc(N4CCOCC4)nc(OCCc4scnc4C)n3)n2)c1]

Compound 7 was prepared by a method analogous to that for Compound 6, except morpholine was used in place of Intermediate III.

LC/MS (mobile phase 5-100% ACN in 5 min), Rt=3.56 min, (M+H)⁺479

### Example 8: Synthesis of 4-((4-((3aR,6aR)-hexahydro-4H-furo[3,2-b]pyrrol-4-yl)-6-(3-(3-methoxyphenyl)-1H-pyrazol-1-yl)pyrimidin-2-yl)oxy)butane-1,2-diol (Compound 8)

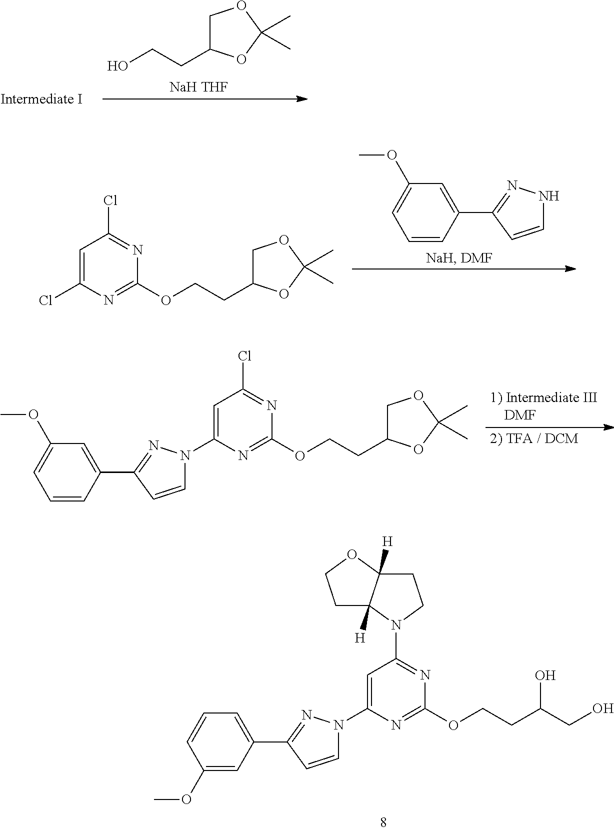

To a solution of Intermediate I, (113 mg, 0.5 mmol) in THF (5 ml) was added NaH (18.4 mg, 0.8 mmol) and the temperature lowered to −78° C. 2-(2,2-dimethyl-1,3-dioxolan-4-yl)ethan-1-ol 71 ul, 0.5 mmol) in THF (1 ml) was added dropwise and the reaction stirred at 1 h at −78° C., worked up by quenching with water, extraction with EtOAc, dried (MgSO₄), filtered and evaporated. Dissolved in THF (3 ml), 3-(3-methoxyphenyl)-1H-pyrazole (18 mg) and NaH (7.5 mg) were added, and the reaction was stirred for 1 h at rt. Intermediate III (35 mg) was added, and the reaction stirred overnight at rt. Quenched with water, evaporated and purified on HPLC, then treated with TFA/DCM 1:2 (0.5 ml), evaporated and purified by LC/MS to give 6 mg of 4-(3-((3aR,6aR)-hexahydro-4H-furo[3,2-b]pyrrol-4-yl)-5-(3-(3-methoxyphenyl)-1H-pyrazol-1-yl)phenoxy)butane-1,2-diol (Compound 8).

LC/MS (mobile phase 5-100% ACN in 5 min), Rt=3.20 min, (M+H)⁺468

### Example 9: Synthesis of 4-((4-(3-(3-methoxyphenyl)-1H-pyrazol-1-yl)-6-morpholinopyrimidin-2-yl)oxy)butane-1,2-diol (Compound 9)

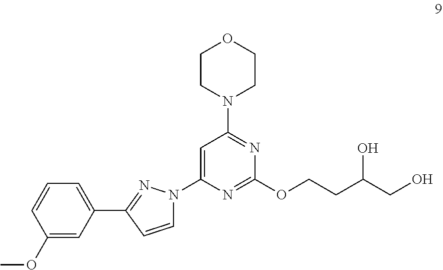 [SMILES: COc1cccc(-c2ccn(-c3cc(N4CCOCC4)nc(OCCC(O)CO)n3)n2)c1]

Compound 9 was prepared by a method analogous to that for Compound 8, except morpholine was used in place of Intermediate III.

LC/MS (mobile phase 5-100% ACN in 5 min), Rt=3.18 min, (M+H)⁺442

### Example 10: Synthesis 4-(6-(4-phenyl-1H-imidazol-1-yl)-2-(2-(pyridin-2-yl)ethoxy)pyrimidin-4-yl)morpholine (Compound 10)

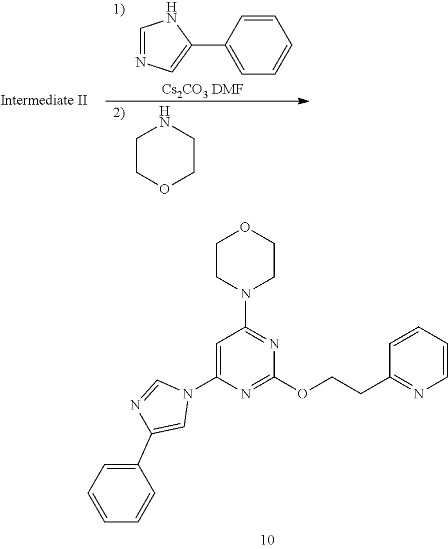

To a solution of Intermediate 11 (27 mg, 0.1 mmol) in DMF (1 ml) was added Cs₂CO₃ (65 mg, 0.2 mmol), followed by 5-phenyl-1H-imidazole (5 mg, 0.1 mmol). The reaction was stirred for 70 min at RT, and morpholine (30 ul) was added and the reaction stirred for 60 min. The mixture was evaporated and purified by LC/MS to give 18 mg of 4-(6-(4-phenyl-1H-imidazol-1-yl)-2-(2-(pyridin-2-yl)ethoxy)pyrimidin-4-yl)morpholine (Compound 10).

LC/MS (mobile phase 5-100% ACN in 5 min), Rt=3.18 min, (M+H)⁺429

### Example 11: Synthesis of (3aR,6aR)-4-(2-((tetrahydrofuran-2-yl)methoxy)-6-(4-(m-tolyl)thiazol-2-yl)pyrimidin-4-yl)hexahydro-2H-furo[3,2-b]pyrrole (Compound 11)

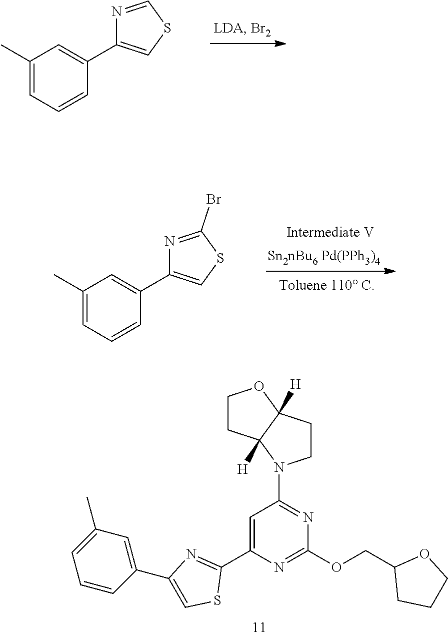 [SMILES: Cc1cccc(-c2cscn2)c1 ; Cc1cccc(-c2csc(Br)n2)c1 ; Cc1cccc(-c2csc(-c3cc(N4CC[C@H]5OCC[C@H]54)nc(OCC4CCCO4)n3)n2)c1]

400 mg of 4-(m-tolyl)thiazole was treated with LDA (1.1 eq) and bromine (1.5 eq) to give 2-bromo-4-(m-tolyl)thiazole. 2-bromo-4-(m-tolyl)thiazole was further converted to (3aR,6aR)-4-(2-((tetrahydrofuran-2-yl)methoxy)-6-(4-(m-tolyl)thiazol-2-yl)pyrimidin-4-yl)hexahydro-2H-furo[3,2-b]pyrrole (Compound 11) as illustrated in the scheme above. 4 mg of product was obtained.

LC/MS (mobile phase 5-100% ACN in 5 min), Rt=3.78 min, (M+H)⁺465

### Example 12: Synthesis of 4-(2-((tetrahydrofuran-2-yl)methoxy)-6-(4-(m-tolyl)thiazol-2-yl)pyrimidin-4-yl)morpholine (Compound 12)

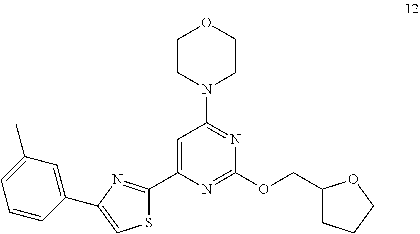 [SMILES: Cc1cccc(-c2csc(-c3cc(N4CCOCC4)nc(OCC4CCCO4)n3)n2)c1]

Compound 12 was prepared by a method analogous to that for Compound 11, except Intermediate VI was added instead of Intermediate V.

LC/MS (mobile phase 5-100% ACN in 3 min), Rt=2.18 min, (M+H)⁺439 ¹H NMR (300 MHz MeOD): δ 8.26 (s, 1H), 7.86 (m, 2H), 7.38 (m, 2H), 7.24 (s, 1H), 3.35 (m, 3H), 3.94 (m, 1H), 3.80 (m, 9H), 2.47 (s, 3H), 2.01 (m, 4H)

### Example 13: Synthesis of 4-(2-((tetrahydrofuran-2-yl)methoxy)-6-(1-(m-tolyl)-1H-pyrazol-3-yl)pyrimidin-4-yl)morpholine (Compound 13)

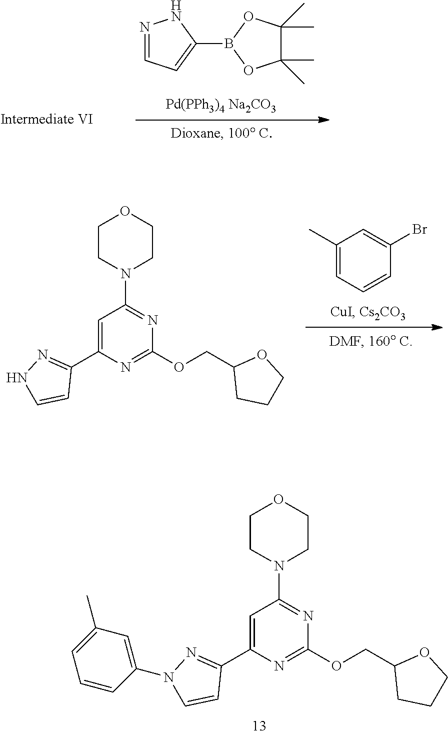

To 200 mg of Intermediate VI in degassed dioxane (10 ml) was added Na₂CO₃ (1.5 eq), Pd(PPh₃)₄ (0.1 eq) and 5-(4,4,5,5-tetramethyl-1,3,2-dioxaborolan-2-yl)-1H-pyrazole (1.3 eq). The reaction was stirred at 100° C. for 2 h under argon. The mixture was evaporated, re-dissolved in EtOAc, washed (saturated aq. sodium bicarbonate), dried (MgSO₄), filtered and evaporated. Re-dissolved in DMF (10 ml), 3-bromotoluene (30 eq), CuI (1 eq) and Cs₂CO₃ (1.1 eq) were added, and the mixture heated at 160° C. for 4 h. The mixture was evaporated, re-dissolved in EtOAc, washed (saturated aq. bicarbonate), dried (MgSO₄), filtered, evaporated, and purified by LC/MS to give 147 mg of Compound 13.

LC/MS (mobile phase 5-100% ACN in 3 min), Rt=1.52 min, (M+H)⁺422 ¹H NMR (300 MHz MeOD): δ 8.28 (s, 1H), 7.71 (s, 1H), 7.65 (m, 1H), 7.39 (m, 1H), 7.21 (d, 1H), 7.11 (m, 2H), 4.39 (m, 3H), 3.93 (m, 1H), 3.78 (m, 9H), 2.49 (s, 3H), 2.00 (m, 4H)

### Example 14: Synthesis of 4-(2-((tetrahydrofuran-2-yl)methoxy)-6-(3-(m-tolyl)-1H-pyrazol-1-yl)pyrimidin-4-yl)-1,4-oxazepane (Compound 14)

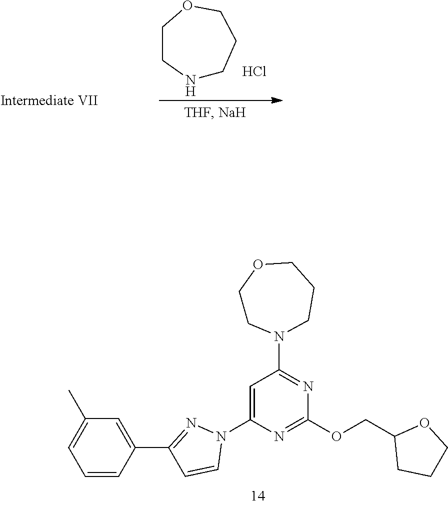 [SMILES: C1CNCCOC1 ; Cl ; Cc1cccc(-c2ccn(-c3cc(N4CCCOCC4)nc(OCC4CCCO4)n3)n2)c1]

To Intermediate VII in THF was added NaH (3.1 eq.) and 1,4-oxepane (3 eq). The mixture was stirred at RT for 2 h. Purified by LC/MS to give Compound 14.

LC/MS (mobile phase 5-100% ACN in 5 min), Rt=3.19 min, (M+H)⁺436 ¹H NMR (300 MHz CDCl₃): δ 8.60 (s, 1H), 7.79 (s, 1H), 7.73 (m, 1H), 7.35 (m, 1H), 7.20 (m, 1H) 6.88 (s, 1H), 6.76 (s, 1H), 4.43 (m, 1H), 4.34 (m, 2H), 3.90 (m, 7H), 3.77 (m, 3H), 2.46 (s, 3H), 2.12 (m, 3H), 1.99 (m, 2H), 1.83 (m, 1H)

### Example 15: Synthesis of 4-(3-methoxypyrrolidin-1-yl)-2-((tetrahydrofuran-2-yl)methoxy)-6-(3-(m-tolyl)-1H-pyrazol-1-yl)pyrimidine (Compound 15)

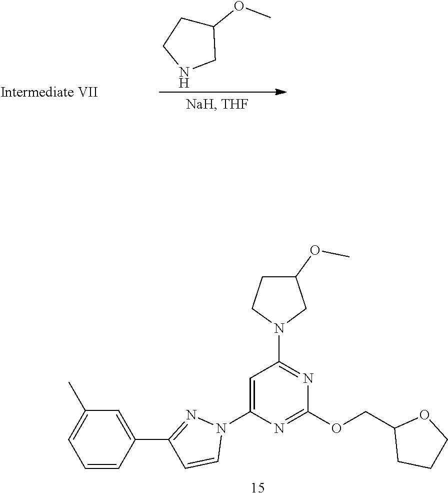

Compound 15 was prepared by a method analogous to that for Compound 14.

LC/MS (mobile phase 5-100% ACN in 5 min), Rt=3.71 min, (M+H)⁺436 ¹H NMR (300 MHz CDCl₃): δ 8.58 (s, 1H), 7.78 (s, 1H), 7.72 (m, 1H), 7.34 (m, 1H), 7.20 (m, 1H), 6.71 (d, 2H), 4.48 (m, 1H), 4.35 (m, 2H), 4.11 (m, 1H), 3.97 (m, 3H), 3.69 (m, 3H), 3.41 (s, 3H), 2.43 (s, 3H), 2.26 (m, 3H), 2.00 (m, 2H), 1.83 (m, 1H)

### Example 16: Synthesis of (3aR,6aR)-4-(6-(3-(3-methoxyphenyl)-1H-pyrazol-1-yl)-2-(2-(pyridin-2-yl)ethoxy)pyrimidin-4-yl)hexahydro-2H-furo[3,2-b]pyrrole (Compound 16)

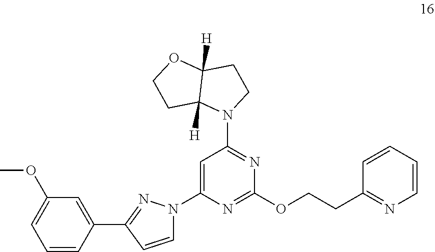 [SMILES: COc1cccc(-c2ccn(-c3cc(N4CC[C@H]5OCC[C@H]54)nc(OCCc4ccccn4)n3)n2)c1]

Compound 16 was prepared by a method analogous to that for Compound 4, except 5-(3-methoxyphenyl)-1H-pyrazole was added instead of 5-phenyl-1H-pyrazole. LC/MS (mobile phase 5-100% ACN in 5 min), Rt=3.89 min, (M+H)⁺485

### Example 17: Synthesis of 2-((tetrahydrofuran-2-yl)methoxy)-4-(tetrahydrofuran-3-yl)-6-(3-(m-tolyl)-1H-pyrazol-1-yl)pyrimidine (Compound 17)

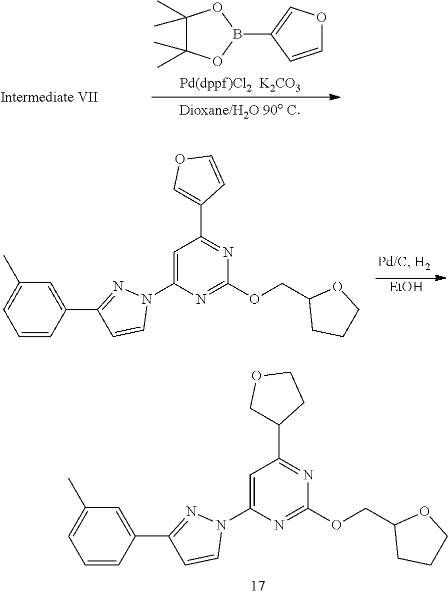 [SMILES: "EtOH" ; CC1(C)OB(c2ccoc2)OC1(C)C ; Cc1cccc(-c2ccn(-c3cc(-c4ccoc4)nc(OCC4CCCO4)n3)n2)c1 ; Cc1cccc(-c2ccn(-c3cc(C4CCOC4)nc(OCC4CCCO4)n3)n2)c1]

To 100 mg of Intermediate VII in degassed dioxane/water 9:1 was added 2-(furan-3-yl)-4,4,5,5-tetramethyl-1,3,2-dioxaborolane (1.2 eq), K₂CO₃ (1.1 eq) and Pd(dppf)Cl₂ (0.3 eq) and the mixture was heated under argon at 90° C. overnight. The mixture was evaporated, re-dissolved in EtOAc and saturated aq. bicarbonate, the layers separated, washed with bicarbonate, dried (MgSO₄), filtered, and evaporated. Re-dissolved in EtOH, Pd/C added and hydrogenated for 4 h at RT. Purified by LC/MS.

LC/MS (mobile phase 5-100% ACN in 3 min), Rt=2.34 min, (M+H)⁺407

¹H NMR (300 MHz CDCl₃): δ 8.62 (s, 1H), 7.79 (s, 1H), 7.73 (d, 1H), 7.61 (s, 1H), 7.35 (m, 1H), 7.24 (m, 1H), 6.81 (s, 1H), 4.45 (m, 3H), 4.22 (m, 1H), 4.12 (m, 1H), 3.85 (m, 1H), 3.61 (m, 1H), 3.39 (m, 2H), 2.49 (s, 3H), 2.11 (m, 3H), 1.83 (m, 1H)

### Example 18: Synthesis of 4-((2-methoxyethyl)thio)-2-((tetrahydrofuran-2-yl)methoxy)-6-(3-(m-tolyl)-1H-pyrazol-1-yl)pyrimidine (Compound 18)

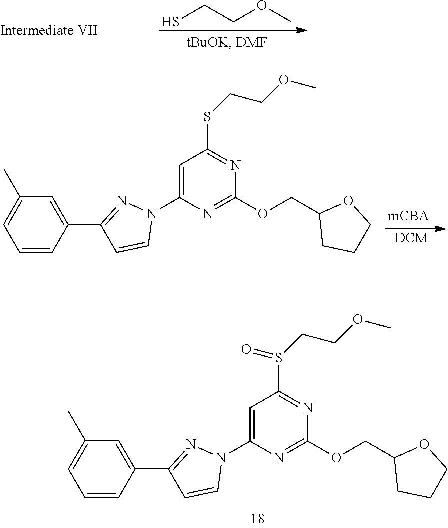

To 60 mg of Intermediate VII in DMF (4 ml) was added tBuOK (1.1 eq) and 2-methoxyethane-1-thiol (1.2 eq) and the reaction stirred at RT overnight. The mixture was evaporated, re-dissolved in EtOAc, washed with sat aq. NaHCO₃, dried (MgSO₄), filtered and evaporated. Re-dissolved in DCM (4 ml) and 1.5 eq of mCPBA added, stirred at RT for 4 h, worked up as described for the last step and purified by LC/MS to give Compound 18.

LC/MS (mobile phase 5-100% ACN in 3 min), Rt=1.92 min, (M+H)⁺443

¹H NMR (300 MHz CDCl₃): δ 8.61 (1H, s), 8.33 (1H, s), 7.82 (1H, s), 7.71 (1H, m), 7.35 (1H, m), 7.24 (1H, m), 6.86 (1H, s), 4.42 (3H, m), 3.90 (4H, m), 3.47 (4H, m), 3.19 (1H, m), 2.48 (3H, s), 2.08 (3H, m), 1.82 (1H, m)

### Example 19: Synthesis of (3aR,6aR)-4-(6-(3-phenyl-1H-pyrazol-1-yl)-2-(2-(pyrimidin-2-yl)ethyl)pyrimidin-4-yl)hexahydro-2H-furo[3,2-b]pyrrole (Compound 19)

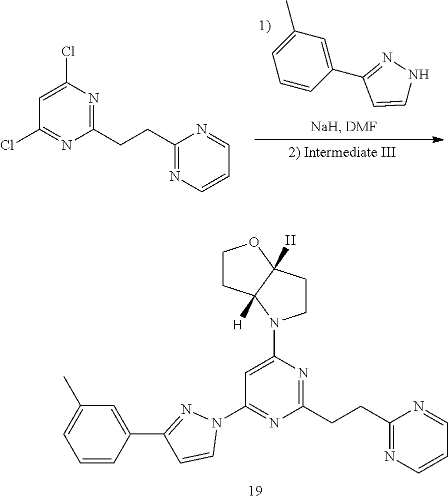 [SMILES: Clc1cc(Cl)nc(CCc2ncccn2)n1 ; Cc1cccc(-c2cc[nH]n2)c1 ; Cc1cccc(-c2ccn(-c3cc(N4CC[C@H]5OCC[C@H]54)nc(CCc4ncccn4)n3)n2)c1]

To 4,6-dichloro-2-(2-(pyrimidin-2-yl)ethyl)pyrimidine (22 mg, 0.09 mmol) in DMF (4 ml) was added 3-(m-tolyl)-1H-pyrazole (47 mg, 0.3 mmol) and NaH (50 mg). The mixture was shaken for 2 h at RT and (3aR,6aR)-hexahydro-412-furo[3,2-b]pyrrole (27 mg, 0.09 mmol) added. The reaction was heated to 80° C. for 4 h and purified by LC/MS to give 7 mg of (3aR,6aR)-4-(6-(3-phenyl-1H-pyrazol-1-yl)-2-(2-(pyrimidin-2-yl)ethyl)pyrimidin-4-yl)hexahydro-2H-furo[3,2-b]pyrrole (Compound 19).

LC/MS (mobile phase 5-100% ACN in 5 min), Rt=4.02 min, (M+H)⁺454

### Example 20: Synthesis of (4-(2-(2-(tetrahydrofuran-2-yl)ethyl)-6-(3-(m-tolyl)-1H-pyrazol-1-yl)pyrimidin-4-yl)morpholin-3-yl)methanol (Compound 20)

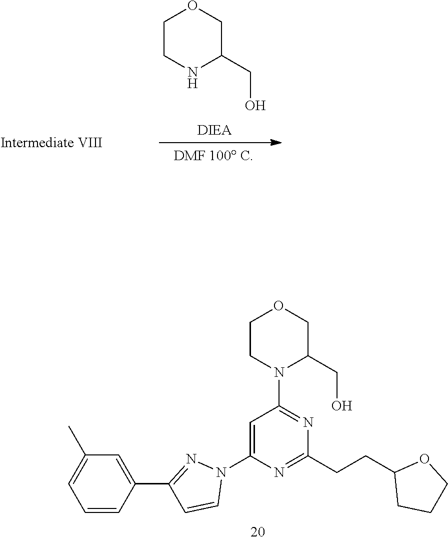 [SMILES: OCC1COCCN1 ; Cc1cccc(-c2ccn(-c3cc(N4CCOCC4CO)nc(CCC4CCCO4)n3)n2)c1]

To 60 mg of Intermediate VIII in DMF (3 ml) was added Cs₂CO₃ (3 eq) and morpholin-3-ylmethanol (3 eq). The reaction was heated at 100 C for 6 h, evaporated and purified by LC/MS to give 22 mg of Compound 20.

LC/MS (mobile phase 5-100% ACN in 3 min), Rt=2.03 min, (M+H)⁺450 ¹H NMR (300 MHz DMSO-d6): δ 8.62 (1H, s), 7.80 (2H, m), 7.36 (1H, m), 7.22 (1H, m), 7.03 (2H, m), 4.99 (1H, m), 4.29 (1H, b), 4.08 (1H, d), 3.95 (1H, m), 3.79 (3H, m), 3.55 (4H, m), 3.76 (2H, m), 2.40 (3H, s), 1.98 (3H, m), 1.82 (2H, m), 1.46 (1H, m)

### Examples 21-90

Compounds 21-90 can be produced using synthetic protocols similar to those described above.

### Example 91: Biological Activity of PIKFYVE Inhibitors

The activity of Compounds 1-20 was measured using a PIKFYVE assay (luciferase ADP-Glo kinase assay available from Promega Corp. of Madison, Wis.). The activity for each compound is provided in Table 1 below (“A” refers to an IC₅₀ of less than 5 nM, “B” refers to an IC₅₀ of 5-100 nM, “C” refers to an IC₅₀ of 101-1,000 nM, and “D” refers to an IC₅₀ of 1,001-10,000 nM).

TABLE 1

| | PIKFYVE Activity |
|---|---|
| Compound | (IC₅₀ in nM) |
| Compound 1 | B |
| Compound 2 | A |
| Compound 3 | A |
| Compound 4 | C |
| Compound 5 | C |
| Compound 6 | B |
| Compound 7 | A |
| Compound 8 | B |
| Compound 9 | A |
| Compound 10 | D |
| Compound 11 | C |
| Compound 12 | B |
| Compound 13 | B |
| Compound 14 | C |
| Compound 15 | D |
| Compound 16 | C |
| Compound 17 | B |
| Compound 18 | D |
| Compound 19 | B |
| Compound 20 | B |

The activity of Compounds 39-90 was also measured using a PIKFYVE assay (luciferase ADP-Glo kinase assay available from Promega Corp. of Madison, Wis.). The activity for each compound is provided in Table 2 below (“1” refers to an IC₅₀ of less than 5 nM, “2” refers to an IC₅₀ of less than 50 nM, “3” refers to an IC₅₀ of less than 500 nM, “4” refers to an IC₅₀ of less than 10 μM, and “ND” refers to an IC₅₀ that was not determined).

TABLE 2

| | PIKFYVE Activity |
|---|---|
| Compound | (IC₅₀ in nM) |
| Compound 39 | 4 |
| Compound 40 | 3 |
| Compound 41 | 4 |
| Compound 42 | 3 |
| Compound 43 | 1 |
| Compound 44 | 1 |
| Compound 45 | 3 |
| Compound 46 | 2 |
| Compound 47 | 2 |
| Compound 48 | 3 |
| Compound 49 | 2 |
| Compound 50 | 1 |
| Compound 51 | 4 |
| Compound 52 | 3 |
| Compound 53 | 3 |
| Compound 54 | 1 |
| Compound 55 | 1 |
| Compound 56 | 1 |
| Compound 57 | 1 |
| Compound 58 | 1 |
| Compound 59 | 2 |
| Compound 60 | 3 |
| Compound 61 | 2 |
| Compound 62 | 1 |
| Compound 63 | 3 |
| Compound 64 | 1 |
| Compound 65 | 1 |
| Compound 66 | 1 |
| Compound 67 | 1 |
| Compound 68 | 1 |
| Compound 69 | 2 |
| Compound 70 | 2 |
| Compound 71 | 1 |
| Compound 72 | 1 |
| Compound 73 | 1 |
| Compound 74 | 1 |
| Compound 75 | 2 |
| Compound 76 | 1 |
| Compound 77 | 2 |
| Compound 78 | 2 |
| Compound 79 | 1 |
| Compound 80 | 1 |
| Compound 81 | 1 |
| Compound 82 | 2 |
| Compound 83 | 3 |
| Compound 84 | 2 |
| Compound 85 | 3 |
| Compound 86 | 1 |
| Compound 87 | 3 |
| Compound 88 | 1 |
| Compound 89 | 1 |
| Compound 90 | 1 |

### Example 92: Pikfyve Inhibition Prevents SARS-CoV-2 Viral Entry

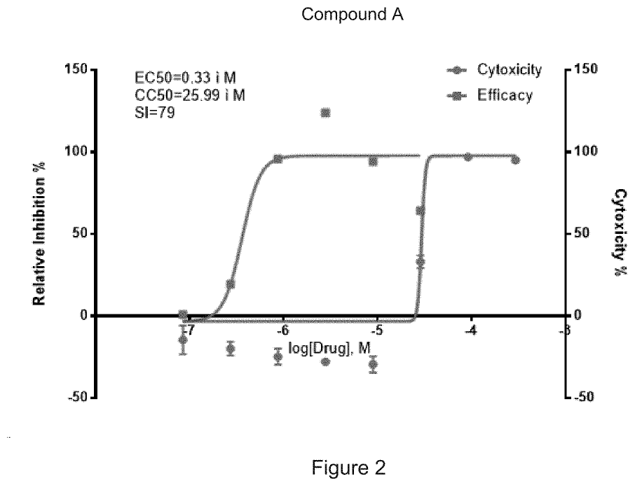

The activity of the compound (S)-2-methoxy-1-(4-morpholino-6-(3-(m-tolyl)-1H-pyrazol-1-yl)pyrimidin-2-yl)ethan-1-ol (Compound A), a Pikfyve inhibitor, was tested for its ability to block live SARS-CoV-2 viral entry in Vero E6 cells. The synthesis of Compound A is described in PCT Application No. PCT/US21/70144, filed Feb. 11, 2021 (Compound 170), which is hereby incorporated by reference. Vero E6 cells lack TMPRSS2 and other proteases and therefore the virus uses the late fusion (endocytosis) pathway in these cells. Vero E6 cells were pre-treated for 1 hour with increasing concentrations of the tested drug and then infected with live SARS-CoV-2 (USA-WA1/2020) at an multiplicity of infection (MOI) of 0.002. On day 5 post-infection, cells were collected for a Natural Red Uptake Assay for an estimate of cell viability. For each condition, units were converted to percentages of uninfected controls using an Excel spreadsheet. Fifty percent virus inhibitory (EC₅₀) or 50% cytotoxicity (CC₅₀) values were determined by nonlinear regression using Prism 6. The results are shown in FIG. 2. The selective index (SI) is the CC₅₀ divided by EC₅₀. The left and right Y-axis of the graphs represent percent relative inhibition of virus induced Clostridium perfringens enterotoxin (CPE) and cytotoxicity of the test drug, respectively. The experiments were done in triplicates.

### Example 93: Efficacy of Antisense Oligonucleotide Suppression of PIKFVE Against SARS-CoV-2 Pseudovirus

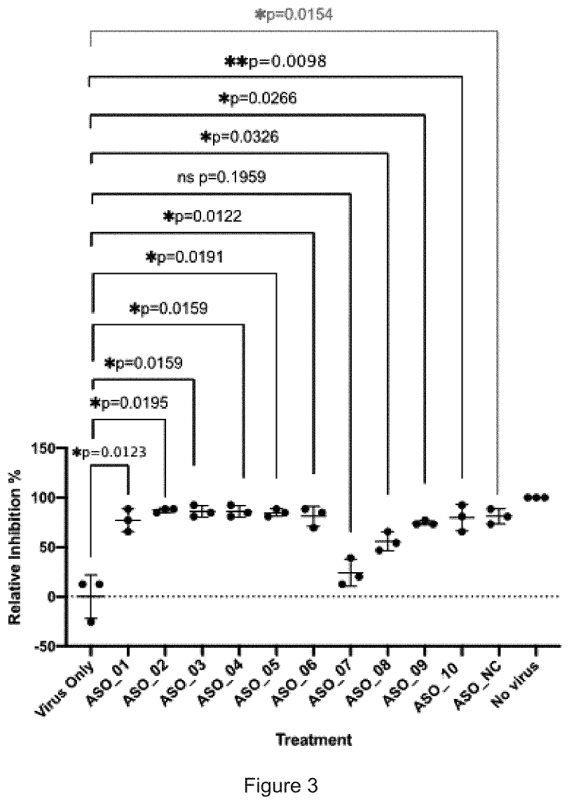

293T cells overexpressing the ACEII receptor were pre-treated for 1 hour with the tested drug (25 nM), in each case one of ten Pikfyve antisense oligonucleotides (ASO-01 to ASO-10) and then infected with SARS-CoV-2 pseudovirus. On day 3 post-infection, cell images were scanned to calculate the GFP+ cells. For each condition, units were converted to percentages of uninfected controls using an Excel spreadsheet. Unparied t-test was performed using Prism 8. The results are provided in FIG. 3. Error bars are the standard derivations for the groups. The Y-axis of the graphs represent percent relative inhibition of virus infection of the drugs.

### Example 94: Efficacy of Combination of PIKFYVE Inhibitor and TMPRSS2 Inhibitor for Virus Using Both Early and Late Fusion Pathways

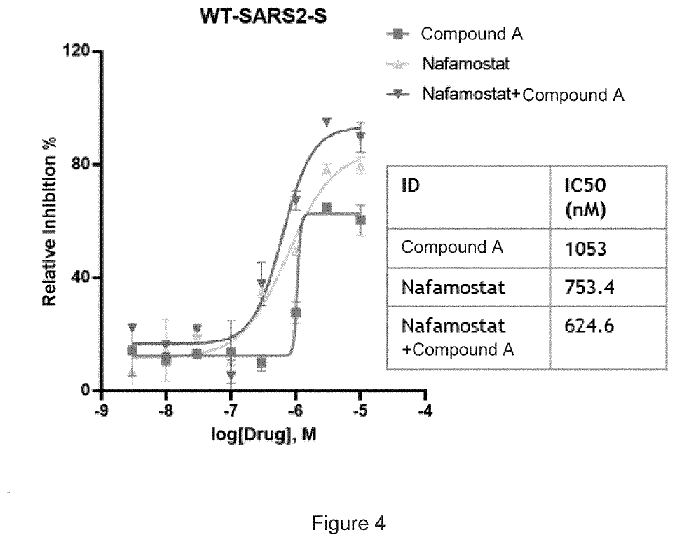

Vero E6 cells overexpressing human ACEII and TMPRSS2 were pre-treated for 1 hour with increasing concentrations of the tested drug (PIKFYVE inhibitor Compound A and TMPRSS2 inhibitor nafamostat) and then infected with SARS-CoV-2 pseudovirus (wildtype SARS2-S). On day 4 post-infection, cell images were scanned to calculate the GFP+ cells. For each condition, units were converted to percentages of uninfected controls using an Excel spreadsheet. Fifty percent virus inhibitory (EC₅₀) values were determined by nonlinear regression using Prism 6. The left and right Y-axis of the graphs represent percent relative inhibition of virus infection of the drugs. The experiments were done in duplicate. The results are shown in FIG. 4. In cells where the virus utilizes both the early and late fusion pathways, the combination of a PIKFYVE inhibitor and a TMPRSS2 inhibitor works better than either therapy along.

All references cited herein are incorporated by reference.

1. A method for treating a subject having a viral infection comprising administering to the subject an effective amount of (i) a PIKfyve inhibitor and (ii) a TMPRSS-2 inhibitor.

2. The method of claim 1, wherein the subject is a human subject.

3. The method of claim 1, wherein the viral infection is a coronavirus infection.

4. The method of claim 3, wherein the coronavirus is SARS-CoV-2.

5. The method of claim 1, wherein the virus is African swine flu, pox virus, Ebola virus, middle east respiratory syndrome virus (MERS), JC polyomavirus (JC), BK polyomavirus (BK), Herpes Simplex Virus (HSV), Marburg virus (MarV), Venezuelan equine encephalitis virus (VEEV), or Lymphocytic choriomeningitis virus (LCMV).

6. The method of claim 1, wherein the PIKfyve inhibitor is apilimod or a pharmaceutically acceptable salt thereof.

7. The method of claim 1, wherein the TMPRSS-2 inhibitor is nafamostat or a pharmaceutically acceptable salt thereof.

8. The method of claim 1, wherein the TMPRSS-2 inhibitor is camostat or a pharmaceutically acceptable salt thereof.

9. A pharmaceutical composition comprising (i) a PIKfyve inhibitor and (ii) a TMPRSS-2 inhibitor.
